(19) United States
(12) Patent Application Publication
(10) Pub. No.: US 2019/0029268 A1
(43) Pub. Date: Jan. 31, 2019

(54) FUNGAL ENDOPHYTE SPECIES

(71) Applicant: Ramot at Tel-Aviv University Ltd., Tel-Aviv (IL)

(72) Inventors: Amir SHARON, Karkur (IL); Yonatan GUR, Tel-Aviv (IL); Maya OFEK-LALZAR, Givataim (IL); Eugenio LLORENS, Borriol (ES); Or SHARON, Tel-Aviv (IL)

(73) Assignee: Ramot at Tel-Aviv University Ltd., Tel-Aviv (IL)

(21) Appl. No.: 16/074,436

(22) PCT Filed: Feb. 2, 2017
(86) PCT No.: PCT/IL2017/050125

§ 371 Date: Aug. 1, 2018

Related U.S. Application Data
(60) Provisional application No. 62/289,959, filed on Feb. 2, 2016.
(60) Provisional application No. 62/312,023, filed on Mar. 23, 2016.

(51) Int. Cl. A01N 63/04; A01H 17/00; A01H 15/00

(52) U.S. Cl. CPC A01N 63/04; A01H 15/00; A01H 17/00

(57) ABSTRACT

A composition of matter comprising an agriculturally acceptable carrier and an endophyte which expresses polynucleotides having the sequences as set forth in SEQ ID NOs: 1-5 or an endophyte which expresses polynucleotides having the sequences as set forth in SEQ ID NOs: 6-10 is disclosed. Uses thereof are also disclosed.

### FIELD AND BACKGROUND OF THE INVENTION

The present invention, in some embodiments thereof, relates to fungal endophyte species and uses thereof.

Recent studies have shown that similar to the situation in humans, the plant microbiome (hereafter termed “phytobiome”) can affect many properties of the host, including tolerance to drought, heat or salt stress, disease susceptibility and vigour. This development, along with a growing need for new agricultural products for plant growth improvement and protection from pests, pathogens and abiotic stresses, has spun great interest in discovery and use of non-synthetic products (collectively called biostimulants), which include growth-promoting and plant-protecting natural compounds as well as beneficial microorganisms.

One class of beneficial microorganisms are endophytes, which are organisms (mainly fungi and bacteria) that live within plants without causing any discernible damage. Beneficial endophytes have been reported in various species of grasses. For example, species that enhance growth of switchgrass for biofuel production (Ghimire and Craven, 2011), species that protect maize from fungal pathogens (Poling et al., 2008), and a species from wild grasses that, when transferred to wheat and tomato, improved growth of these plants under heat and salt stress conditions (Redman et al., 2002; Rodriguez et al., 2008). Similarly, a Trichoderma species has been developed as a commercial biostimulant product for maize and other crops. Endophytes have been described also in cultivated wheat, with positive effect on drought and heat tolerance, as well as enhanced resistance to soil-borne pathogens (Crous et al., 1995; Hubbard et al., 2012; Larran et al., 2002; Marshall et al., 2000; Waller et al., 2005).

Background art includes Mei and Flinn, Recent Patents on Biotechnology 2010, 4, pages 81-95, European Patent Application EP2521442, International Patent Application WO2012174585A1, US Patent Application No. 20150373993, U.S. Pat. No. 7,232,565 and U.S. Pat. No. 6,815,591.

### SUMMARY OF THE INVENTION

According to an aspect of the present invention there is provided a composition of matter comprising an agriculturally acceptable carrier and an endophyte which expresses polynucleotides having the sequences as set forth in SEQ ID NOs: 1-5 or an endophyte which expresses polynucleotides having the sequences as set forth in SEQ ID NOs: 6-10.

According to an aspect of the present invention there is provided a composition of matter comprising an agriculturally acceptable carrier and an endophyte deposited under the NRRL deposit No. 67222 or 67223.

According to an aspect of the present invention there is provided an article of manufacture comprising an isolated endophyte which expresses polynucleotides having the sequences as set forth in SEQ ID NOs: 1-5 or an endophyte which expresses polynucleotides having the sequences as set forth in SEQ ID NOs: 6-10 and an agent which promotes the growth of a plant.

According to an aspect of the present invention there is provided an article of manufacture comprising an isolated endophyte which is deposited under the NRRL deposit No. 67222 and 67223 and an agent which promotes the growth of a plant.

According to an aspect of the present invention there is provided a composition of matter comprising an extract of an endophyte which expresses polynucleotides having the sequences as set forth in SEQ ID NOs: 1-5 or an endophyte which expresses polynucleotides having the sequences as set forth in SEQ ID NOs: 6-10.

According to an aspect of the present invention there is provided a composition of matter comprising an extract of an endophyte which is deposited under the NRRL deposit No. 67222 or 67223.

According to an aspect of the present invention there is provided a plant or part thereof comprising an isolated endophyte which expresses polynucleotides having the sequences as set forth in SEQ ID NOs: 1-5 or an endophyte which expresses polynucleotides having the sequences as set forth in SEQ ID NOs: 6-10, wherein the plant is not a wild grass.

According to an aspect of the present invention there is provided a plant or part thereof comprising an isolated endophyte which is deposited under the NRRL deposit No. 67222 or 67223.

According to an aspect of the present invention there is provided a method of enhancing the growth of a plant comprising:

(a) inoculating the plant or a part thereof with an endophyte which expresses polynucleotides having the sequences as set forth in SEQ ID NOs: 1-5 or an endophyte which expresses polynucleotides having the sequences as set forth in SEQ ID NOs: 6-10; and

(b) growing the plant, thereby improving the growth of the plant.

According to an aspect of the present invention there is provided a method of providing a plant tolerance to a stressful condition comprising:

(a) inoculating the plant or a part thereof with an endophyte which expresses polynucleotides having the sequences as set forth in SEQ ID NOs: 1-5 or an endophyte which expresses polynucleotides having the sequences as set forth in SEQ ID NOs: 6-10; and

(b) growing the plant, thereby providing the plant tolerance to a stressful condition.

According to an aspect of the present invention there is provided a method of increasing nutrient uptake in a plant comprising:

(a) inoculating the plant or a part thereof with an endophyte which expresses polynucleotides having the sequences as set forth in SEQ ID NOs: 1-5 or an endophyte which expresses polynucleotides having the sequences as set forth in SEQ ID NOs: 6-10; and

(b) growing the plant, thereby increasing nutrient uptake in the plant.

According to an aspect of the present invention there is provided a method of enhancing the growth of a plant comprising:

(a) inoculating the plant or a part thereof with an endophyte deposited under the NRRL deposit No. 67222 or 67223; and

(b) growing the plant, thereby improving the growth of the plant.

According to an aspect of the present invention there is provided a method of providing a plant tolerance to a stressful condition comprising:

(a) inoculating the plant or a part thereof with an endophyte deposited under the NRRL deposit No. 67222 or 67223; and

(b) growing the plant, thereby providing a plant tolerance to a stressful condition.

According to an aspect of the present invention there is provided a method of increasing nutrient uptake in a plant comprising:

(a) inoculating the plant or a part thereof with an endophyte deposited under the NRRL deposit No. 67222 or 67223; and

(b) growing the plant, thereby increasing nutrient uptake in the plant.

According to embodiments of the present invention, the endophyte which expresses polynucleotides having the sequences as set forth in SEQ ID NOs: 1-5 is deposited under the NRRL deposit No. 67223.

According to embodiments of the present invention, the endophyte which expresses polynucleotides having the sequences as set forth in SEQ ID NOs: 6-10 is deposited under the NRRL deposit No. 67222.

According to embodiments of the present invention, the endophyte is in the form of a spore, a hyphae, or a mycelia.

According to embodiments of the present invention, the composition further comprises at least one agent which promotes the growth of a plant.

According to embodiments of the present invention, the at least one agent is selected from the group consisting of an antibacterial agent, an insecticide and a nematocide.

According to embodiments of the present invention, the at least one agent is a pesticide.

According to embodiments of the present invention, the composition further comprises a fertilizer.

According to embodiments of the present invention, the endophyte is viable.

According to embodiments of the present invention, the endophyte which expresses polynucleotides having the sequences as set forth in SEQ ID NOs: 1-5 is deposited under the NRRL deposit No. 67223.

According to embodiments of the present invention, the endophyte which expresses polynucleotides having the sequences as set forth in SEQ ID NOs: 6-10 is deposited under the NRRL deposit No. 67222.

According to embodiments of the present invention, the endophyte is in the form of a spore, a hyphae, or a mycelia.

According to embodiments of the present invention, the agent is selected from the group consisting of an antibacterial agent, an insecticide and a nematocide.

According to embodiments of the present invention, the agent is a fertilizer.

According to embodiments of the present invention, the endophyte is viable.

According to embodiments of the present invention, the endophyte which expresses polynucleotides having the sequences as set forth in SEQ ID NOs: 1-5 is deposited under the NRRL deposit No. 67223.

According to embodiments of the present invention, the endophyte which expresses polynucleotides having the sequences as set forth in SEQ ID NOs: 6-10 is deposited under the NRRL deposit No. 67222.

According to embodiments of the present invention, the extract comprises at least one volatile organic compound of the endophyte.

According to embodiments of the present invention, the endophyte which expresses polynucleotides having the sequences as set forth in SEQ ID NOs: 1-5 is deposited under the NRRL deposit No. 67223.

According to embodiments of the present invention, the endophyte which expresses polynucleotides having the sequences as set forth in SEQ ID NOs: 6-10 is deposited under the NRRL deposit No. 67222.

According to embodiments of the present invention, the isolated endophyte is present at a concentration of at least about 250 CFU or spores per seed.

According to embodiments of the present invention, the endophyte which expresses polynucleotides having the sequences as set forth in SEQ ID NOs: 1-5 is deposited under the NRRL deposit No. 67223.

According to embodiments of the present invention, the endophyte which expresses polynucleotides having the sequences as set forth in SEQ ID NOs: 6-10 is deposited under the NRRL deposit No. 67222.

According to embodiments of the present invention, the endophyte is in the form of a spore, a hyphae or a mycelia.

According to embodiments of the present invention, the part of the plant is selected from the group consisting of a root, a bulb, a seed, a seedling, a leaf, a flower and a branch.

According to embodiments of the present invention, the part of the plant is a seed.

According to embodiments of the present invention, the growing is effected under water limiting conditions.

According to embodiments of the present invention, the growing is effected under a stressful condition.

According to embodiments of the present invention, the stressful condition is an abiotic stress.

According to embodiments of the present invention, the abiotic stress is selected from the group consisting of drought, heat, cold, salt stress and low nutrient stress.

According to embodiments of the present invention, the method further comprises analyzing the growth of the plant.

According to embodiments of the present invention, the method further comprises harvesting the plant.

According to embodiments of the present invention, the method further comprises selecting the plant.

According to embodiments of the present invention, the plant is a crop plant.

According to embodiments of the present invention, the plant is a cultivated crop plant.

According to embodiments of the present invention, the cultivated crop plant is wheat.

According to embodiments of the present invention, the plant is a monocot.

According to embodiments of the present invention, the plant is a dicot.

According to embodiments of the present invention, the plant is selected from the group consisting of wheat, corn, soybean, rice and sugarcane.

According to embodiments of the present invention, the enhancing the growth comprises at least one of increasing the plant height, increasing the plant fresh weight, increasing the number of plant shoots, increasing the plant dry weight and increasing the plant crop yield.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are bar graphs illustrating the occurrence of fungal endophytes in Triticum aestivum (TA), T. dicoccoides (TD) and Aegilops sharonensis (AS) plant stems (P) seeds (S) and stems of F1 plants (F1). A) Percentage of plants in which culturable fungal endophytes were detected (E+). B) Percentage of E+plants in which more than one fungal OTU (≥97% sequence similarity) was detected. * Significant difference (χ², P<0.05).

FIGS. 2A-B are Venn diagrams showing shared and unique fungal endophytic OTUs across A) sample types for each plant species or B) Plant species for each sample type. The numbers of core OTUs (centre of each diagram) in each comparison are indicated within the brackets.

FIG. 3 is a bar graph illustrating the composition of endophytic fungal community. Prevalence (%) of the six most frequent OTUs detected in the fungal endophytes culture collection.

FIGS. 4A-B are graphs comparing endopytic fungal community colonizing stems of A. sharonensis and T. dicoccoides (n=6) assessed by cultivation-independent ITS sequencing approach. ITS sequences were grouped into operative taxonomic units (OTUs) based on 97% sequence similarity. A) Venn diagram depicting shared and unique OTUs detected in each of the plant species. The numbers in parenthesis represent shared and uniquely detected OTUs with relative abundance >0.5%. B) non-metric multidimensional scaling ordination, based on Bray-Cutris similarity matrix between the endofungal community profiles (Stress=0.086).

FIG. 5 is a heatmap presenting relative abundance of the key prevalent ciOTUs detected in AS and TD stems. Significant differences in relative abundance between the plant species (FDR adjusted P value) as well as association to cultivated isolates based on sequence similarity (97%) are presented.

FIG. 6 illustrates photographs of plants under 100 mM NaCl conditions.

FIG. 7 is a bar graph illustrating plant height under normal (0) and salt (100 mM and 200 mM NaCl) conditions.

FIG. 8 is a bar graph illustrating root length under normal (0) and salt (100 mM and 200 mM NaCl) conditions.

FIG. 9 is a bar graph illustrating shoot biomass under normal (0) and salt (100 mM and 200 mM NaCl) conditions.

FIG. 10 is a bar graph illustrating root biomass under normal (0) and salt (100 mM and 200 mM NaCl) conditions.

FIG. 11 is a bar graph illustrating leaf width under normal (0) and salt (100 mM and 200 mM NaCl) conditions.

FIG. 12 is a bar graph illustrating chlorophyll content under normal (0) and salt (100 mM and 200 mM NaCl) conditions.

FIG. 13 is a photograph of plants under water-limiting conditions.

FIG. 14 is a bar graph illustrating plant height under normal (0) and water limiting (drought) conditions.

FIG. 15 is a bar graph illustrating root length under normal (0) and water limiting (drought) conditions.

FIG. 16 is a bar graph illustrating shoot biomass under normal (0) and water limiting (drought) conditions.

FIG. 17 is a bar graph illustrating root biomass under normal (0) and water limiting (drought) conditions.

FIG. 18 is a bar graph illustrating leaf width under normal (0) and water limiting (drought) conditions.

FIG. 19 is a bar graph illustrating leaf area under normal (0) and water limiting (drought) conditions.

FIG. 20 is a photograph of wheat plants grown in large containers in sand.

FIG. 21A is a bar graph illustrating the weight and number of tillers of plants grown in the field.

FIGS. 21B-G are bar graphs summarizing the yield data from samples taken at maturation.

FIG. 22 is a photograph illustrating the increased number of tillers of plants grown in pots in the greenhouse.

FIG. 23A is a photograph illustrating seedlings 14 days after sowing.

FIGS. 23B-C are bar graphs summarizing the data of the germination rate of the wheat seeds after sowing.

FIGS. 24A-B are bar graphs illustrating 24A) Relative water content and 24B) membrane stability index in leaves of wheat inoculated with Acremonium sclerotigenum or Sarocladium implicatum under control or water limiting conditions.

FIGS. 25A-B are bar graphs illustrating accumulation of proline in 25A) leaves and 25B) roots of wheat inoculated with Acremonium sclerotigenum or Sarocladium implicatum under control or water limiting conditions.

FIG. 26 is a bar graph illustrating accumulation of MDA in leaves of wheat inoculated with Acremonium sclerotigenum or Sarocladium implicatum under control or water limiting conditions.

FIGS. 27A-B are bar graphs illustrating the levels of 27A) Abscisic acid and 27B) Jasmonic isoleucine measured in leaves of wheat inoculated with Acremonium sclerotigenum or Sarocladium implicatum under control or water limiting conditions.

FIGS. 28A-B are bar graphs illustrating levels of Phenolic compounds in wheat under control or water limiting conditions. 28A) Caffeic acid, 28B) Ferulic acid.

FIG. 29 is a photograph illustrating the symptoms of plants after 10 days under drought stress.

FIG. 30 is a bar graph summarizing the dry weight of Arabidopsis vegetative part under drought stress.

FIGS. 31A-D are bar graphs illustrating levels of Abscisic acid, Jasmonic isoleucine, Jasmonic acid, and Caffeic levels in A. thaliana leaves inoculated with Acremonium sclerotigenum (13237) or Sarocladium implicatum (14005) under control or water limiting conditions.

FIG. 32 is a photograph illustrating the results of a PCR analysis illustrating successful infection of crop plants with the Acremonium isolate.

FIGS. 33A-B are bar graphs illustrating shoot and root fresh weight of maize seedlings.

FIG. 34 are images showing GFP-labelled fungi growing in the leaf tissue and emerging from the stoma. Lower images show enlargement of the parts marked with a rectangle in the top images.

### DESCRIPTION OF SPECIFIC EMBODIMENTS OF THE INVENTION

The present invention, in some embodiments thereof, relates to fungal endophyte species and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors isolated fungal endophytes from wild grasses relatives of wheat: isolate 13237 was obtained from Aegilops sharonensis (Sharon goat grass), isolate 14005 was obtained from Tritcum dicoccoides (wild wheat). Taxonomy of the two endophytes was determined based on sequence of four genes. Isolate 13237 was classified as Acremonium sclerotigenum, isolate 14005 was classified as Sarocladium implicatum. Additional endophytes were also isolated.

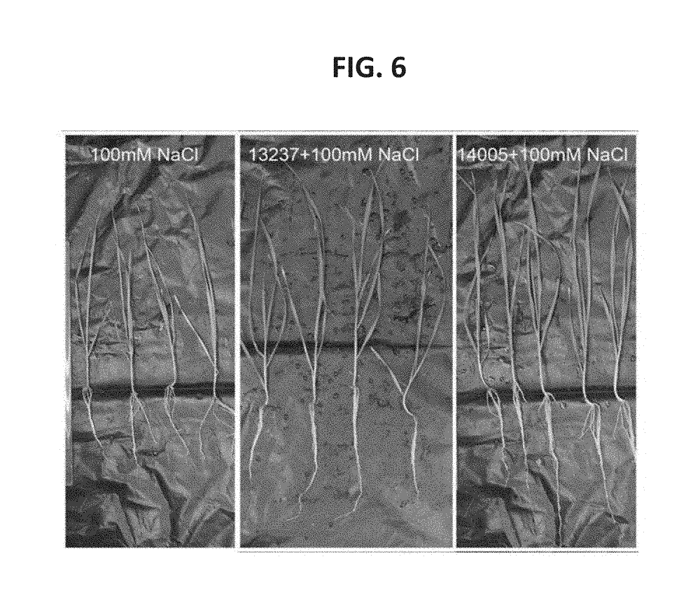

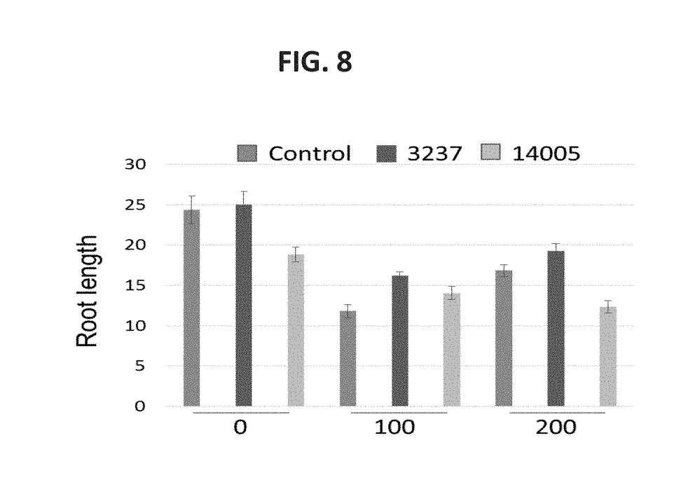

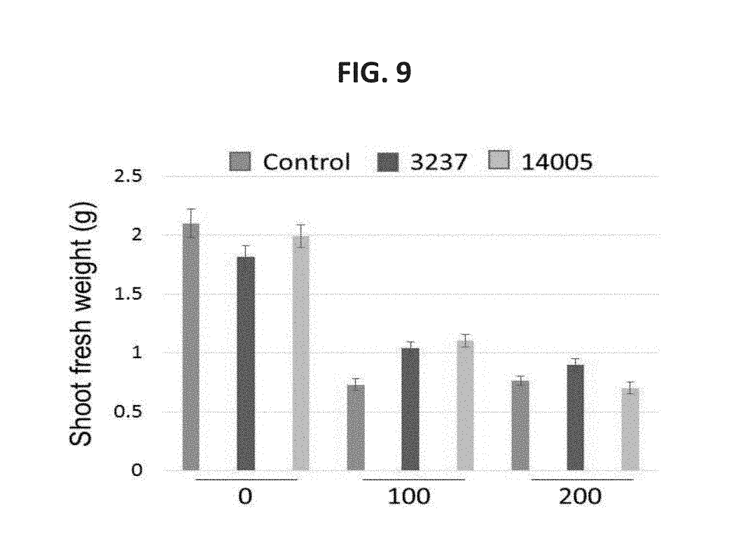

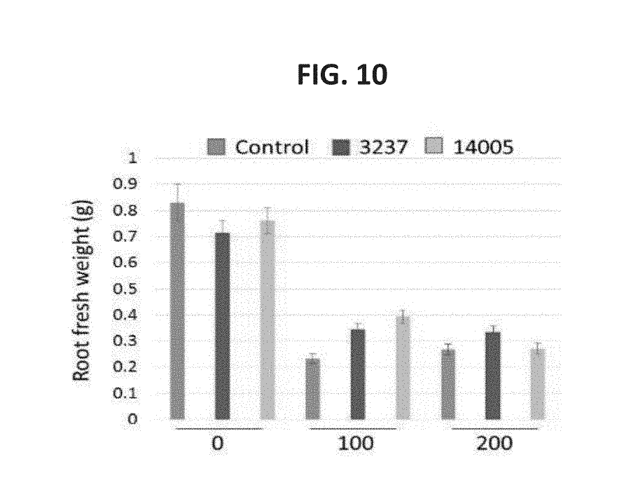

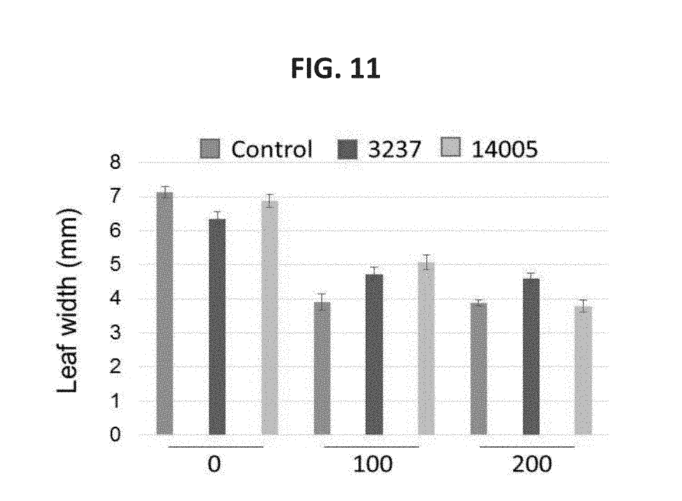

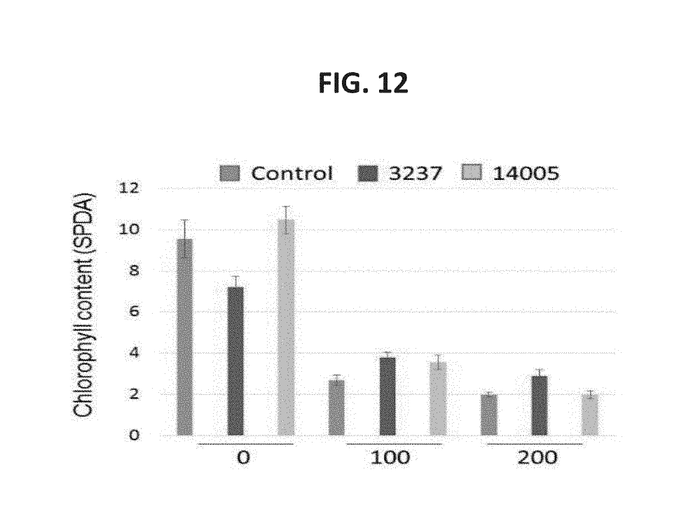

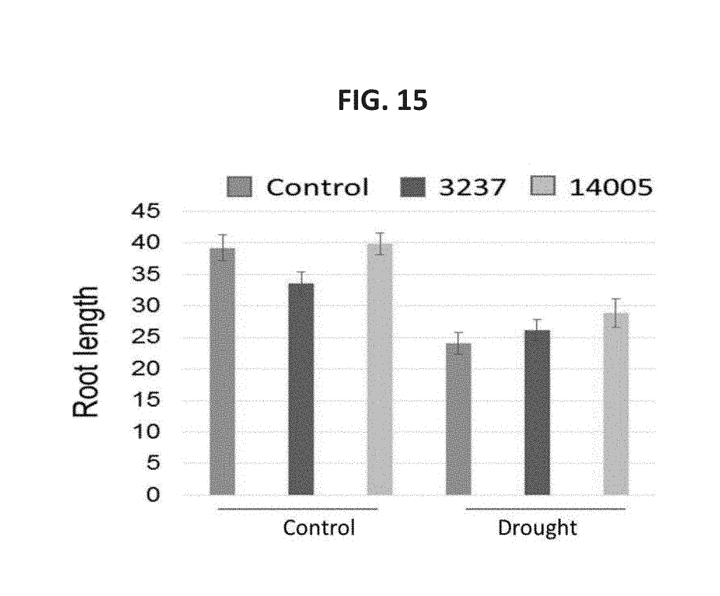

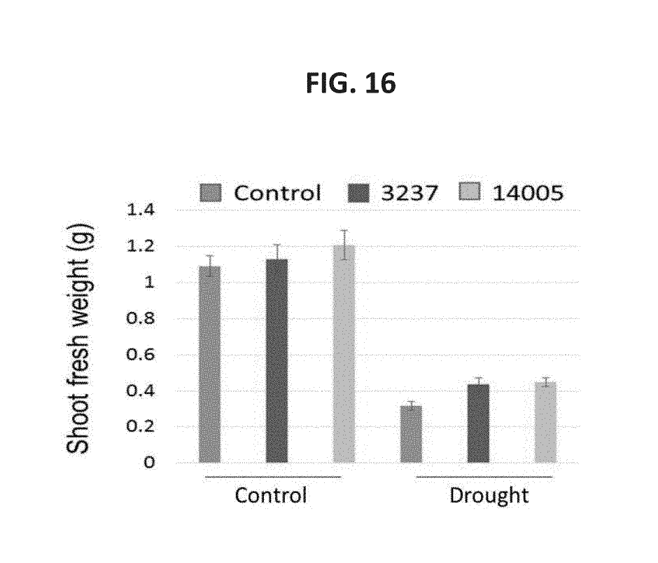

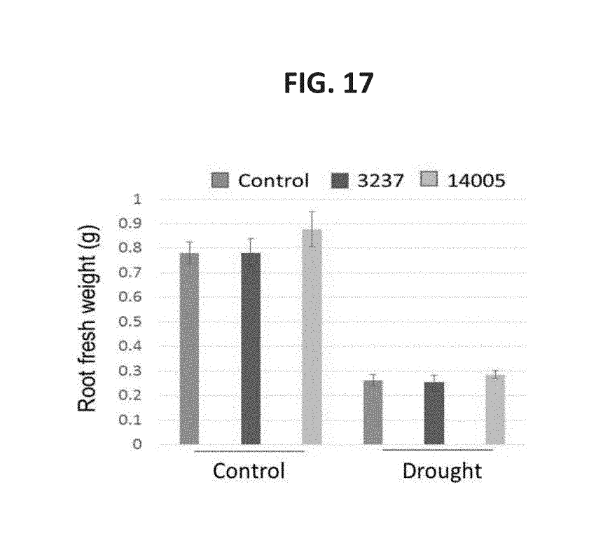

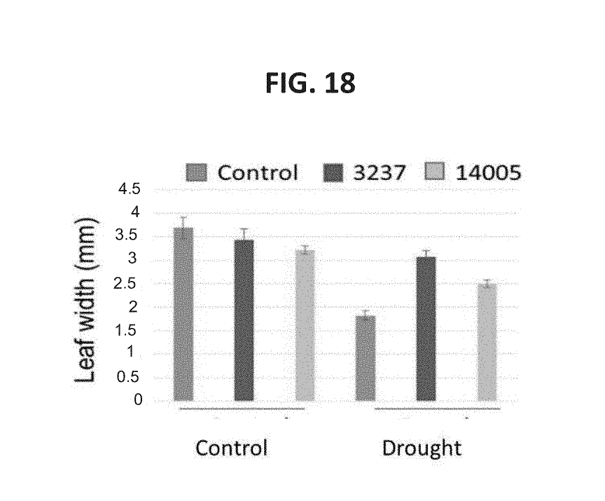

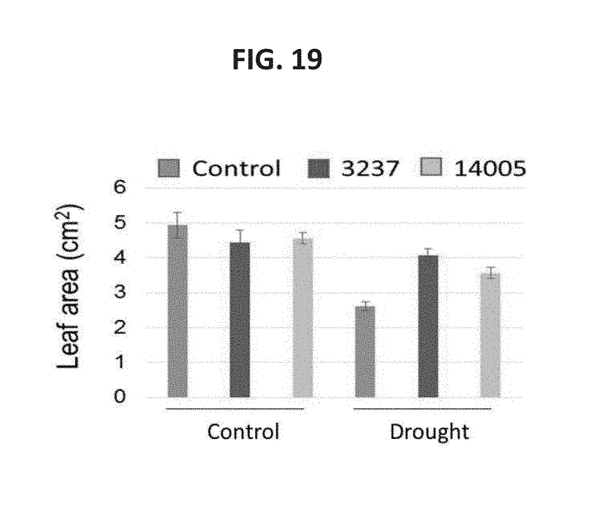

In order to determine the effect of these fungal species on cultivated plants, wheat seeds were inoculated with the fungal spores and plant performance was evaluated in greenhouse experiments under optimal conditions, in water containing 100 mM and 200 mM NaCl (salt stress), and under water limiting conditions (drought stress). Under all conditions, the endophytes-inoculated plants performed better than control wheat plants that were not inoculated with the endophytes. Improved growth parameters included significantly higher shoot and root biomass, taller plants, and retention of higher levels of chlorophyll under salt stress (FIGS. 6-19). The present inventors propose that the isolated endophytes may be used to improve sustainability and yield in crop plants in general and wheat, in particular.

Thus, according to a first aspect of the present invention, there is provided an isolated endophyte which is deposited in NRRL deposit Nos. 67222 or 67223.

According to a first embodiment, the endophyte expresses genes having sequences as set forth in SEQ ID NOs: 1-5.

According to another embodiment, the endophyte expresses genes having sequences as set forth in SEQ ID NOs: 6-10.

The present inventors contemplate any other endophyte strain/species that expresses at least 3, at least 4 or at least 5 of the genes having at least 97%, 98%, 99%, 99.5%, 99.6%, 99.7%, 99.8% or 99.9% homology to any of the sequences as set forth in SEQ ID NOs: 1-5.

Furthermore, the present inventors contemplate any other endophyte strain/species that expresses at least 3, at least 4 or at least 5 of the genes having at least 97%, 98%, 99%, 99.5%, 99.6%, 99.7%, 99.8% or 99.9% homology to any of the sequences as set forth in SEQ ID NOs: 6-10.

As used herein, the term “endophyte” refers to an organism capable of living within a plant or is otherwise associated therewith, and does not cause disease or harm the plant otherwise (i.e. is capable of living symbiotically with the plant). Endophytes can occupy the intracellular or extracellular spaces of plant tissue, including the leaves, stems, flowers, fruits, seeds, or roots. An endophyte can be for example a bacterial or fungal organism.

According to a particular embodiment, the endophyte is a filamentous fungus and belongs to the family of non-clavicipitaceous fungi.

The term “isolated” is intended to specifically reference an organism, cell, tissue, polynucleotide, or polypeptide that is removed from its original source and purified from additional components with which it was originally associated. For example, an endophyte may be considered isolated from removed and purified from a plant or plant element so that it is isolated and no longer associated with its source plant or plant element.

The endophyte may be present as a spore, a hyphae, or a mycelia.

In one embodiment, the endophyte is stored such that it is propagatable. For example, the endophyte may be dried (e.g. freeze-dried) or frozen. In another embodiment, the endophyte is in a culture. Media for propagating endophyte may include soil, hydroponic apparatus, and/or artificial growth medium.

In still another embodiment, an extract of the endophyte is envisaged. The extract may comprise volatile organic compounds and/or metabolites of the endophytes which have growth promoting properties.

The endophyte of this aspect of the present invention may be formulated with an agriculturally acceptable carrier.

In some embodiments, the agricultural carrier may be soil or plant growth medium. Other agricultural carriers that may be used include fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, leaf, root, plant elements, sugar cane bagasse, hulls or stalks from grain processing, ground plant material or wood from building site refuse, sawdust or small fibers from recycling of paper, fabric, or wood. Other suitable formulations will be known to those skilled in the art.

In one embodiment, the formulation can comprise additives, including but not limited to sticking agents, spreading agents, surfactants, synergists, penetrants, compatibility agents, buffers, acidifiers, defoaming agents, thickeners and drift retardants.

In another embodiment, the formulation can comprise a tackifier or adherent. Such agents are useful for combining the endophyte of the invention with carriers that can contain other compounds (e.g., control agents that are not biologic), to yield a coating composition. Such compositions may aid to maintain contact between the endophyte and a plant or plant part. In one embodiment, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers. Other examples of adherent compositions that can be used in the synthetic preparation include those described in EP 0818135, CA 1229497, WO 2013090628, EP 0192342, WO 2008103422 and CA 1041788, each of which is incorporated herein by reference in its entirety.

The formulation may also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant. Such desiccants are ideally compatible with the endophytic population used, and should promote the ability of the endophytic population to survive application on the plants or parts thereof and to survive desiccation. Examples of suitable desiccants include one or more of trehalose, sucrose, glycerol, and Methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In one embodiment, the formulation comprises a fertilizer. Preferably, the fertilizer is one that does not reduce the viability of the endophyte by more than 20%, 30%, 40%, 50% or more.

In some cases, it is advantageous for the formulation to contain agents such as an antibacterial agent, an herbicide, a nematicide, an insecticide, a plant growth regulator, a rodenticide, and a nutrient. Such agents are ideally compatible with the plant onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

In liquid form, (for example, solutions or suspensions), the endophytes of the present invention can be mixed or suspended in aqueous solutions. Suitable liquid diluents or carriers include aqueous solutions, petroleum distillates, or other liquid carriers.

Solid compositions can be prepared by dispersing the endophytes of the present invention in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

The formulations comprising the endophytes of the present invention typically contains between about 0.1 to 95% by weight, for example, between about 1% and 90%, between about 3% and 75%, between about 5% and 60%, between about 10% and 50% in wet weight of the endophytic population of the present invention. It is preferred that the formulation contains at least about 10² CFU or spores per ml of formulation, at least about 10³ CFU or spores per ml of formulation, at least about 10⁴ CFU or spores per ml of formulation, at least about 10⁵ CFU or spores per ml of formulation, at least about 10⁶ CFU or spores per ml of formulation, or at least about 10⁷ CFU or spores per ml of formulation.

The present inventors also contemplate that the presently disclosed endophytes may be comprised in an article of manufacture which further comprises an agent which promotes the growth of plants.

The agents may be formulated together with the endophytes in a single composition, or alternatively packaged separately, but in a single container.

Suitable agents are described herein above. Other suitable agents include fertilizers, pesticides (e.g. an antibacterial agent, an herbicide, a nematocide, a fungicide an insecticide), a plant growth regulator, a rodenticide, and a nutrient, as further described herein below.

In one embodiment, the agent which promotes the growth of the plant lacks fungicidal activity.

In another embodiment, the agent which promotes the growth of the plant is a fungicide.

Exemplary fungicides contemplated by the present invention include but are not limited to respiration inhibitors such as, inhibitors of complex III at Q 0 site (e.g. strobilurins): azoxystrobin, coumethoxystrobin, coumoxystrobin, dimoxystrobin, enestroburin, fenaminstrobin, fenoxystrobin/flufenoxystrobin, fluoxastrobin, Isofetamid, kresoxim-methyl, metominostrobin, orysastrobin, picoxystrobin, pyraclostrobin, pyrametostrobin, pyraoxystrobin, trifloxystrobin, 2-[2-(2,5-dimethyl-phenoxymethyl)-phenyl]-3-methoxy-acrylic acid methyl ester and 2-(2-(3-(2,6-di-chlorophenyl)-1-methyl-allylideneaminooxymethyl)-phenyl)-2-methoxyimino-N-methyl-acetamide, pyribencarb, triclopyricarb/chlorodincarb, famoxadone, fenamidone; inhibitors of complex III at Q, site: cyazofamid, amisulbrom, [(3S,6S,7R,8R)-8-benzyl-3-[(3-acetoxy-4-methoxy-pyridine-2-carbonyl)amino]-6-methyl-4,9-dioxo-1,5-dioxonan-7-yl] 2-methylpropanoate, [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl] 2-methylpropanoate, [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl] 2-methylpropanoate, [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl] 2-methylpropanoate; (3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate; Inhibitors of complex II (e. g. carboxamides): benodanil, benzovindiflupyr, bixafen, boscalid, carboxin, fenfuram, fluopyram, flutolanil, fluxapyroxad, furametpyr, isofetamid, isopyrazam, mepronil, oxycarboxin, penflufen, penthiopyrad, sedaxane, tecloftalam, thifluz-amide, N-(4′-trifluoromethylthiobiphenyl-2-yl)-3-difluoromethyl-1-methyl-1H-pyrazole-4-carboxamide, N-(2-(1,3,3-trimethyl-butyl)-phenyl)-1,3-dimethyl-5-fluoro-1H-pyrazole-4-carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 1,3,5-tri-methyl-N-(1,1,3-trimethylindan-4-yl)pyrazole-4-carboxamide, N-(7-fluoro-1,1,3-trimethyl-indan-4-yl)-1,3-dimethyl-pyrazole-4-carboxamide, N-[2-(2,4-dichlorophenyl)-2-methoxy-1-methyl-ethyl]-3-(difluoromethyl)-1-methyl-pyrazole-4-carboxamide; other respiration inhibitors (e.g. complex I, uncouplers): diflumetorim, (5,8-difluoro-quinazolin-4-yl)-{2-[2-fluoro-4-(4-trifluoromethylpyridin-2-yloxy)-phenyl]-ethyl}-amine.

Other fungicides contemplated by the present invention include nitrophenyl derivates (such as binapacryl, dinobuton, dinocap, fluazinam; ferimzone; organometal compounds: fentin salts, such as fentin-acetate, fentin chloride or fentin hydroxide; ametoctradin; and silthiofam).

Other fungicides contemplated by the present invention include sterol biosynthesis inhibitors (SBI fungicides) including C14 demethylase inhibitors (DMI fungicides): triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-[re/-(2S;3f?)-3-(2-chlorophenyl)-2-(2,4-difluorophenyl)-oxiranylmethyl]-5-thiocyanato-1H-[1,2,4]triazole, 2-[re/-(2S;3R)-3-(2-chlorophenyl)-2-(2,4-difluorophenyl)-oxiranylmethyl]-2H-[1,2,4]triazole-3-thiol; imidazoles: imazalil, pefurazoate, prochloraz, triflumizol.

Other fungicides contemplated by the present invention include pyrimidines, pyridines and piperazines: fenarimol, nuarimol, pyrifenox, triforine; 3-(4-chloro-2-fluoro-phenyl)-5-(2,4-difluorophenyl)isoxazol-4-yl]-(3-pyridyl)methanol.

Other fungicides contemplated by the present invention include delta14-reductase inhibitors, such as aldimorph, dodemorph, dodemorph-acetate, fenpropimorph, tridemorph, fenpropidin, piperalin, spiroxamine.

Other fungicides contemplated by the present invention include inhibitors of 3-keto reductase such as fenhexamid.

Phenylamides or acyl amino acid fungicides include benalaxyl, benalaxyl-M, kiralaxyl, metalaxyl, metalaxyl-M (mefenoxam), ofurace, oxadixyl.

Other contemplated fungicides include hymexazole, octhilinone, oxolinic acid, bupirimate, 5-fluorocytosine, 5-fluoro-2-(p-tolylmethoxy)pyrimidin-4-amine, 5-fluoro-2-(4-fluorophenylmethoxy)pyrimidin-4-amine; and Inhibitors of cell division and cytoskeleton.

Other contemplated fungicides include tubulin inhibitors, such as benzimidazoles, thiophanates: benomyl, carbendazim, fuberidazole, thiabendazole, thiophanate-methyl; triazolopynmidines: 5-chloro-7-(4-methyl-piperidin-1-yl)-6-(2,4,6-trifluorophenyl)-[1,2,4]triazolo[1,5-a]pyrimidine.

Other contemplated fungicides include cell division inhibitors such as diethofencarb, ethaboxam, pencycuron, fluopicolide, zoxamide, metrafenone, pyriofenone.

Other contemplated fungicides include inhibitors of amino acid and protein synthesis such as methionine synthesis inhibitors (anilino-pyrimidines): cyprodinil, mepanipyrim and pyrimethanil.

Protein synthesis inhibitors include, but are not limited to blasticidin-S, kasugamycin, kasugamycin hydrochloride-hydrate, mildiomycin, streptomycin, oxytetracyclin, polyoxine, and validamycin A.

Signal transduction inhibitors: include MAP/histidine kinase inhibitors: fluoroimid, iprodione, procymidone, vinclozolin, fenpiclonil and fludioxonil;

G protein inhibitors include quinoxyfen.

Lipid and membrane synthesis inhibitors include but are not limited to phospholipid biosynthesis inhibitors such as edifenphos, iprobenfos, pyrazophos and isoprothiolane.

Other contemplated fungicides include those involved in lipid peroxidation such as dicloran, quintozene, tecnazene, tolclofos-methyl, biphenyl, chloroneb and etridiazole.

Other contemplated fungicides include those involved in phospholipid biosynthesis and cell wall deposition such as dimethomorph, flumorph, mandipropamid, pyrimorph, benthiavalicarb, iprovalicarb, valifenalate and N-(1-(1-(4-cyano-phenyl)ethanesulfonyl)-but-2-yl) carbamic acid-(4-fluorophenyl) ester.

Other contemplated fungicides include compounds affecting cell membrane permeability and fatty acids: propamocarb, propamocarb-hydrochloride. Fatty acid amide hydrolase inhibitors: oxathiapiprolin, 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, 2-{3-[2-(1-{[3,5-bis(difluoromethyl-1H-pyrazol-1-yl]acetyl}piperidin-4-yl)-1,3-thiazol-4-yl]-4,5-dihydro-1,2-oxazol-5-yl}phenyl methanesulfonate, 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate.

Other contemplated fungicides include inhibitors with multi-site action. These include inorganic active substances such as Bordeaux mixture, copper acetate, copper hydroxide, copper oxychloride, basic copper sulfate, sulfur; thio- and dithiocarbamates: ferbam, mancozeb, maneb, metam, metiram, propineb, thiram, zineb, ziram; organochlorine compounds (e.g. phthalimides, sulfamides, chloronitriles): anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methyl-benzenesulfonamide.

Guanidines are also contemplated by the present invention—these include guanidine, dodine, dodine free base, guazatine, guazatine-acetate, iminoctadine, iminoctadine-triacetate, iminoctadine-tris(albesilate), dithianon, 2,6-dimethyl-1H,5H-[1,4]dithiino[2,3-c:5,6-c′]dipyrrole-1,3,5,7(2H,6H)-tetraone.

Other contemplated fungicides include cell wall synthesis inhibitors. These include inhibitors of glucan synthesis such as validamycin, polyoxin B; melanin synthesis inhibitors: pyroquilon, tricyclazole, carpropamid, dicyclomet, fenoxanil.

Other contemplated fungicides include plant defense inducers such as acibenzolar-S-methyl, probenazole, isotianil, tiadinil, prohexadione-calcium; phosphonates: fosetyl, fosetyl-aluminum, phosphorous acid and its salts.

Other contemplated fungicides include bronopol, chinomethionat, cyflufenamid, cymoxanil, dazomet, debacarb, diclomezine, difenzoquat, difenzoquat-methylsulfate, diphenylamin, fenpyrazamine, flumetover, flusulfamide, flutianil, methasulfocarb, nitrapyrin, nitrothal-isopropyl, oxin-copper, proquinazid, tebufloquin, tecloftalam, oxathiapiprolin, 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-{5-[2-chloro-6-(prop-2-yn-1-yl-oxy)phenyl]-4,5-dihydro-1,2-oxazol-3-yl}-1,3-thiazol-2-yl)piperidin-1-yl]ethanone, tolprocarb, triazoxide, 2-butoxy-6-iodo-3-propylchromen-4-one, 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-{5-[2-chloro-6-(prop-2-yn-1-yl-oxy)phenyl]-4,5-dihydro-1,2-oxazol-3-yl}-1,3-thiazol-2-yl)piperidin-1-yl]ethanone, N-(cyclo-propylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2-methyl-4-(3-trimethylsilanyl-propoxy)-phenyl)-N-ethyl-N-methyl formamidine, 2methoxy-acetic acid 6-tert-butyl-8-fluoro-2,3-dimethyl-quinolin-4-yl ester, 3-[5-(4-methylphenyl)-2,3-dimethyl-isoxazolidin-3-yl]-pyridine, 3-[5-(4-chloro-phenyl)-2,3-dimethyl-isoxazolidin-3-yl]-pyridine (pyrisoxazole), N-(6-methoxy-pyridin-3-yl) cyclopropanecarboxylic acid amide, 5-chloro-1-(4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide; ethyl (Z)-3-amino-2-cyano-3-phenyl-prop-2-enoate, tert-butyl N-[6-[[(Z)-[(1-methyltetrazol-5-yl)-phenyl-methylene]amino]oxymethyl]-2-pyridyl]carbamate, pentyl N-[6-[[(Z)-[(1-methyltetrazol-5-yl)-phenyl-methylene]amino]oxymethyl]-2-pyridyl]carbamate, 2-[2-[(7,8-difluoro-2-methyl-3-quinolyl)oxy]-6-fluoro-phenyl]propan-2-ol, 2-[2-fluoro-6-[(8-fluoro-2-methyl-3-quinolyl)oxy]phenyl]propan-2-ol, 3-(5-fluoro-3,3,4,4-tetramethyl-3,4-dihydroisoquinolin-1-yl)quinoline, 3-(4,4-difluoro-3,3-dimethyl-3,4-dihydroisoquinolin-1-yl)quinoline, 3-(4,4,5-trifluoro-3,3-dimethyl-3,4-dihydroisoquinolin-1-yl)quinolone; 9-fluoro-2,2-dimethyl-5-(3-quinolyl)-3H-1,4-benzoxazepine, ethyl (Z)-3-amino-2-cyano-3-phenyl-prop-2-enoate, picarbutrazox, pentyl N-[6-[[(Z)-[(1-methyltetrazol-5-yl)-phenyl-methylene]amino]oxymethyl]-2-pyridyl]carbamate, 2-[2-[(7,8-difluoro-2-methyl-3-quinolyl)oxy]-6-fluoro-phenyl]propan-2-ol, 2-[2-fluoro-6-[(8-fluoro-2-methyl-3-quinolyl)oxy]phen-yl]propan-2-ol, 3-(5-fluoro-3,3,4,4-tetramethyl-3,4-dihydroisoquinolin-1-yl)quinoline, 3-(4,4-difluoro-3,3-dimethyl-3,4-dihydroisoquinolin-1-yl)quinoline, 3-(4,4,5-trifluoro-3,3-dimethyl-3,4-dihydroisoquinolin-1-yl)quinolone.

In another embodiment, the agent which promotes the growth of the plant is a biopesticide.

Exemplary biopesticides include Microbial pesticides with fungicidal, bactericidal, viricidal and/or plant defense activator activity such as Ampelomyces quisqualis, Aspergillus flavus, Aureobasidium pullulans, Bacillus amyloliquefaciens, B. mojavensis, B. pumilus, B. simplex, B. solisalsi, B. subtilis, B. subtilis var. amyloliquefaciens, Candida oleophila, C. saitoana, Clavibacter michiganensis (bacteriophages), Coniothyrium minitans, Cryphonectria parasitica, Cryptococcus albidus, Dilophosphora alopecuri, Fusarium oxysporum, Clonostachys rosea f. catenulate (also named Gliocladium catenulatum), Gliocladium roseum, Lysobacter antibioticus, L. enzymogenes, Metschnikowia fructicola, Microdochium dimerum, Microsphaeropsis ochracea, Muscodor albus, Paenibacillus polymyxa, Pantoea vagans, Phlebiopsis gigantea, Pseudomonas sp., Pseudomonas chloraphis, Pseudozyma flocculosa, Pichia anomala, Pythium oligandrum, Sphaerodes mycoparasitica, Streptomyces griseoviridis, S. lydicus, S. violaceusniger, Talaromyces flavus, Trichoderma asperellum, T. atroviride, T. fertile, T. gamsii, T. harmatum, T. harzianum; mixture of T. harzianum and T. viride; mixture of T. polysporum and T. harzianum; T. stromaticum, T. virens (also named Gliocladium virens), T. viride, Typhula phacorrhiza, Ulocladium oudemansii, Verticillium dahlia, zucchini yellow mosaic virus (avirulent strain).

Exemplary biochemical pesticides with fungicidal, bactericidal, viricidal and/or plant defense activator activity include chitosan (hydrolysate), harpin protein, laminarin, Menhaden fish oil, natamycin, Plum pox virus coat protein, potassium or sodium bicarbonate, Reynoutria sachlinensis extract, salicylic acid, tea tree oil.

Microbial pesticides with insecticidal, acaricidal, molluscidal and/or nematicidal activity: include but are not limited to Agrobacterium radiobacter, Bacillus cereus, B. firmus, B. thuringiensis, B. thuringiensis ssp. aizawai, B. t. ssp. israelensis, B. t. ssp. galleriae, B. t. ssp. kurstaki, B. t. ssp. tenebrionis, Beauveria bassiana, B. brongniartii, Burkholderia sp., Chromobacterium subtsugae, Cydia pomonella granulosis virus, Cryptophlebia leucotreta granulovirus (CrleGV), Isaria fumosorosea, Heterorhabditis bacteriophora, Lecanicillium longisporum, L. muscarium (formerly Verticillium lecanii), Metarhizium anisopliae, M. anisopliae var. acridum, Nomuraea rileyi, Paecilomyces fumosoroseus, P. lilacinus, Paenibacillus popilliae, Pasteuria spp., P. nishizawae, P. penetrans, P. ramose, P. reneformis, P. thornea, P. usgae, Pseudomonas fluorescens, Steinernema carpocapsae, S. feltiae, S. kraussei.

Biochemical pesticides with insecticidal, acaricidal, molluscidal, pheromone and/or nematicidal activity include L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, cis-jasmone, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, potassium silicate, sorbitol actanoate, (E,Z,Z)-3,8,11-tetradecatrienyl acetate, (Z,E)-9,12-tetradecadien-1-yl acetate, Z-7-tetradecen-2-one, Z-9-tetradecen-1-yl acetate, Z-11-tetradecenal, Z-11-tetradecen-1-ol, Acacia negra extract, extract of grapefruit seeds and pulp, extract of Chenopodium ambrosiodae, Catnip oil, Neem oil, Quillay extract and Tagetes oil.

Microbial pesticides with plant stress reducing, plant growth regulator, plant growth promoting and/or yield enhancing activity: Azospirillum amazonense, A. brasilense, A. lipoferum, A. irakense, A. halopraeferens, Bradyrhizobium sp., B. elkanii, B. japonicum, B. liaoningense, B. lupini, Delftia acidovorans, Glomus intraradices, Mesorhizobium sp., Paenibacillus alvei, Penicillium bilaiae, Rhizobium leguminosarum bv. phaseoli, R. I. trifolii, R. I. bv. viciae, R. tropici and Sinorhizobium meliloti.

Biochemical pesticides with plant stress reducing, plant growth regulator and/or plant yield enhancing activity: abscisic acid, aluminium silicate (kaolin), 3-decen-2-one, formononetin, genistein, hesperetin, homobrassinlide, humates, jasmonic acid or salts or derivatives thereof, lysophosphatidyl ethanolamine, naringenin, polymeric polyhydroxy acid, Ascophyllum nodosum (Norwegian kelp, Brown kelp) extract and Ecklonia maxima (kelp) extract.

In one embodiment, the agent which promotes the growth of the plant is a herbicide.

Below is a list of exemplary herbicides contemplated by the present invention.

Acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor.

Amino acid derivatives: bilanafos, glyphosate, glufosinate, sulfosate;

Aryloxyphenoxypropionates: clodinafop, cyhalofop-butyl, fenoxaprop, fluazifop, haloxyfop, metamifop, propaquizafop, quizalofop, quizalofop-P-tefuryl;

Bipyridyls: diquat, paraquat;

(Thio)carbamates: asulam, butylate, carbetamide, desmedipham, dimepiperate, eptam (EPTC), esprocarb, molinate, orbencarb, phenmedipham, prosulfocarb, pyributicarb, thiobencarb, triallate;

Cyclohexanediones: butroxydim, clethodim, cycloxydim, profoxydim, sethoxydim, tepraloxydim, tralkoxydim;

Dinitroanilines: benfluralin, ethalfluralin, oryzalin, pendimethalin, prodiamine, trifluralin; diphenyl ethers: acifluorfen, aclonifen, bifenox, diclofop, ethoxyfen, fomesafen, lactofen, oxyfluorfen;

Hydroxybenzonitriles: bomoxynil, dichlobenil, ioxynil;

Imidazolinones: imazamethabenz, imazamox, imazapic, imazapyr, imazaquin, imazethapyr;

Phenoxy acetic acids: clomeprop, 2,4-dichlorophenoxyacetic acid (2,4-D), 2,4-DB, dichlorprop, MCPA, MCPA-thioethyl, MCPB, Mecoprop;

Pyrazines: chloridazon, flufenpyr-ethyl, fluthiacet, norflurazon, pyridate;

Pyridines: aminopyralid, clopyralid, diflufenican, dithiopyr, fluridone, fluroxypyr, picloram, picolinafen, thiazopyr;

Sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metazosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxy-pyrimidin-2-yl)urea;

Triazines: ametryn, atrazine, cyanazine, dimethametryn, ethiozin, hexazinone, metamitron, metribuzin, prometryn, simazine, terbuthylazine, terbutryn, triaziflam;

Ureas: chlorotoluron, daimuron, diuron, fluometuron, isoproturon, linuron, metha-benzthiazuron, tebuthiuron;

Other acetolactate synthase inhibitors: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam; others: amicarbazone, aminotriazole, anilofos, beflubutamid, benazolin, bencarbazone, benfluresate, benzofenap, bentazone, benzobicyclon, bicyclopyrone, bromacil, bromobutide, butafenacil, butamifos, cafenstrole, carfentrazone, cinidon-ethyl, chlorthal, cinmethylin, clomazone, cumyluron, cyprosulfamide, dicamba, difenzoquat, diflufenzopyr, Drechslera monoceras, endothal, ethofumesate, etobenzanid, fenoxasulfone, fentrazamide, flumiclorac-pentyl, flumioxazin, flupoxam, flurochloridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester.

In one embodiment, the agent which promotes the growth of the plant is an insecticide.

Examples of insecticides contemplated by the present invention are described herein below.

Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;

Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;

Pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;

Insect growth regulators: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb;

Lipid biosynthesis inhibitors: spirodiclofen, spiromesifen, spirotetramat;

Nicotinic receptor agonists/antagonists compounds: clothianidin, dinotefuran, flupyradifurone, imidacloprid, thiamethoxam, nitenpyram, acetamiprid, thiacloprid, 1-2-chloro-thiazol-5-ylmethyl)-2-nitrimino-3,5-dimethyl-[1,3,5]triazinane;

GABA antagonist compounds: endosulfan, ethiprole, fipronil, vaniliprole, pyrafluprole, pyriprole, 5-amino-1-(2,6-dichloro-4-methyl-phenyl)-4-sulfinamoyl-1H-pyrazole-3-carbothioic acid amide;

Macrocyclic lactone insecticides: abamectin, emamectin, milbemectin, lepimectin, spinosad, spinetoram;

Mitochondrial electron transport inhibitor (METI) I acaricides: fenazaquin, pyridaben, tebufenpyrad, tolfenpyrad, flufenerim;

METI II and III compounds: acequinocyl, fluacyprim, hydramethylnon;

Uncouplers: chlorfenapyr;

Oxidative phosphorylation inhibitors: cyhexatin, diafenthiuron, fenbutatin oxide, propargite;

Moulting disruptor compounds: cryomazine;

Mixed function oxidase inhibitors: piperonyl butoxide;

Sodium channel blockers: indoxacarb, metaflumizone;

Ryanodine receptor inhibitors: chlorantraniliprole, cyantraniliprole (former cyazypyr), flubendiamide, N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide; N-[4-chloro-2-[(diethyl-lambda-4-sulfanylidene)carbamoyl]-6-methyl-phenyl]-2-(3-chloro-2-pyridyl)-5-(triflu-oromethyl)pyrazole-3-carboxamide; N-[4-chloro-2-[(di-2-propyl-lambda-4-sulfanyli-dene)carbamoyl]-6-methyl-phenyl]-2-(3-chloro-2-pyridyl)-5-(trifluoromethyl)pyrazole-3-carboxamide; N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide; N-[4,6-dichloro-2-[(diethyl-lambda-4-sulfanylidene)carbamoyl]-phenyl]-2-(3-chloro-2-pyridyl)-5-(difluoromethyl)pyrazole-3-carboxamide; N-[4,6-dibromo-2-[(di-2-propyl-lambda-4-sulfanylidene)carbamoyl]-phenyl]-2-(3-chloro-2-pyridyl)-5-(trifluoromethyl)pyrazole-3-carboxamide; N-[4-chloro-2-[(di-2-propyl-lambda-4-sulfanylidene)carbamoyl]-6-cyano-phenyl]-2-(3-chloro-2-pyridyl)-5-(trifluoromethyl)pyrazole-3-carboxamide; N-[4,6-dibromo-2-[(diethyl-lambda-4-sulfanylidene)carbamoyl]-phenyl]-2-(3-chloro-2-pyridyl)-5-(tri-fluoromethyl)pyrazole-3-carboxamide;

Others: benclothiaz, bifenazate, cartap, flonicamid, pyridalyl, pymetrozine, sulfur, thiocyclam, cyenopyrafen, flupyrazofos, cyflumetofen, amidoflumet, imicyafos, bistrifluron, pyrifluquinazon and 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-tri methyl-11-oxo-9-(3-pyridinyl)-2H,11H-naphtho[2,1-b]pyrano[3,4-e]pyran-3,6-diyl] cyclopropaneacetic acid ester.

In one embodiment, the agent which promotes the growth of the plant is an antibacterial agent.

Antibacterial Agents:

Exemplary antibacterial agents include streptomycin, oxytetracycline, oxolinic acid, or gentamicin.

In one embodiment, the agent which promotes the growth of the plant is a plant growth regulator.

Plant Growth Regulators:

In one embodiment, the plant growth regulator is selected from the group consisting of: abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, trinexapac-ethyl and uniconazole;

In one embodiment, the agent which promotes the growth of the plant is a nematocide.

Nematocides:

Exemplary nematocides include but are not limited to cadusafos, dichlofenthion, ethoprophos, fenamiphos, fluensulfone, fosthiazate, fosthietan, imicyafos, isamidofos, isazofos, methyl bromide, methyl isothiocyanate, oxamyl, sodium azide, BYI-1921 (experimental name) and MAI-08015 (experimental name).

In one embodiment, the agent which promotes the growth of the plant is a nutrient.

Nutrients:

In another embodiment, the article of manufacture can comprise a nutrient. The nutrient can be selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, Potassium nitrate. Such compositions can exist as free salts or ions within the seed coat composition. Alternatively, nutrients/fertilizers can be complexed or chelated to provide sustained release over time.

In one embodiment, the agent that promotes the growth of the plant is a rodenticide.

Rodenticides:

Rodents such as mice and rats cause considerable economical damage by eating and soiling planted or stored seeds. Moreover, mice and rats transmit a large number of infectious diseases such as plague, typhoid, leptospirosis, trichinosis and salmonellosis. Anticoagulants such as coumarin and indandione derivatives play an important role in the control of rodents. These active ingredients are simple to handle, relatively harmless to humans and have the advantage that, as the result of the delayed onset of the activity, the animals being controlled identify no connection with the bait that they have ingested, therefore do not avoid it. This is an important aspect in particular in social animals such as rats, where individuals act as tasters. In one embodiment, the article of manufacture may comprise a rodenticide selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide.

As mentioned, the endophytes of the present invention may be used to enhance the growth of host plants.

Thus, according to another aspect of the present invention there is provided a method of enhancing the growth of a plant comprising:

(a) inoculating the plant or a part thereof with at least one of the disclosed endophytes; and

(b) growing the plant, thereby improving the growth of the plant.

The phrase “improving the growth” as used herein refers to enhancing the rate of growth and/or amount of the plant, or a component thereof (such as a seed, leaf, fruit, stem etc.) as compared to a plant grown under identical conditions, but in the absence of the endophyte.

Thus, for example, the present inventors contemplate that the endophytes of the present invention may be used to enhance the germination of the seeds of the plant.

In another embodiment, the endophytes of the present invention increase the number and/or size of the seeds of the plant.

In another embodiment, the endophytes of the present invention increase the number and/or size of the shoots of plant—(i.e. a tillering effect).

In another embodiment, the endophytes of the present invention increase the amount of fruit and/or size and/or weight of the fruit produced by the plant.

In another embodiment, the endophytes of the present invention increase the amount of foliage produced by the plant.

In another embodiment, the endophytes of the present invention increase the height the plant.

In another embodiment, the endophytes of the present invention increase the weight of the plant.

The growth enhancing effects of the endophytes of the present invention may be apparent under stressful conditions or non-stressful conditions.

Thus, the endophyte may enhance the growth of a plant under a stressful condition as compared to the growth of the plant under that identical condition but grown in the absence of the endophyte.

In one embodiment, growing the plant in the presence of the endophyte provides tolerance to a stressful condition.

Exemplary stressful conditions under which the plant may be grown include, but are not limited to abiotic stress conditions including drought conditions, heat, cold or salt stress, low nutrient stress and other stressful conditions such as stress induced by other plants (e.g. weeds, cultivated or native plants).

The plants of this aspect of the invention may be grown in areas which are prone to stressful conditions. Alternatively, or additionally, the plants of this aspect of the invention may be grown at times of year which are stressful to the plants.

In one embodiment, growing the plant in the presence of the endophyte enhances plant nutrient uptake.

According to a particular embodiment, the plants which are inoculated are agricultural plants.

The phrase “agricultural plants”, or “plants of agronomic importance”, refers to plants that are cultivated by humans for food, feed, fiber, and fuel purposes. In one embodiment, the plant is not a wild plant.

In one embodiment, a monocotyledonous plant is used. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In a particular embodiment, the monocotyledonous plant can be selected from the group consisting of a maize, rice, wheat, oats, barley and sugarcane.

In another embodiment, a dicotyledonous plant is used, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In a particular embodiment, the dicotyledonous plant can be selected from the group consisting of cotton, soybean, pepper, and tomato.

Preferably, the plant is an agricultural plant. Agricultural plants include monocotyledonous species such as: maize (Zea mays), common wheat (Triticum aestivum), spelt (Triticum spelta), einkorn wheat (Triticum monococcum), emmer wheat (Triticum dicoccum), durum wheat (Triticum durum), Asian rice (Oryza sativa), African rice (Oryza glabaerreima), wild rice (Zizania aquatica, Zizania latifolia, Zizania palustris, Zizania texana), barley (Hordeum vulgare), Sorghum (Sorghum bicolor), Finger millet (Eleusine coracana), Proso millet (Panicum miliaceum), Pearl millet (Pennisetum glaucum), Foxtail millet (Setaria italica), Oat (Avena sativa), Triticale (Triticosecale), rye (Secale cereal), Russian wild rye (Psathyrostachys juncea), bamboo (Bambuseae), or sugarcane (e.g., Saccharum arundinaceum, Saccharum barberi, Saccharum bengalense, Saccharum edule, Saccharum munja, Saccharum officinarum, Saccharum procerum, Saccharum ravennae, Saccharum robustum, Saccharum sinense, or Saccharum spontaneum); as well as dicotyledonous species such as: soybean (Glycine max), canola and rapeseed cultivars (Brassica napus), cotton (genus Gossypium), alfalfa (Medicago sativa), cassava (genus Manihot), potato (Solanum tuberosum), tomato (Solanum lycopersicum), pea (Pisum sativum), chick pea (Cicer arietinum), lentil (Lens culinaris), flax (Linum usitatissimum) and many varieties of vegetables. In a particular embodiment, the agricultural plant is a cereal.

In a particular embodiment, plants contemplated for inoculation by the present inventors include wheat, corn, soybean, rice and sugarcane.

A “host plant” refers to any plant, particularly a plant of agronomic importance, which the endophytes of the present invention can colonize. The endophyte is the to “colonize” a plant or seed when it can be stably detected within the plant or seed over a period time, such as one or more days, weeks, months or years, in other words, a colonizing entity is not transiently associated with the plant or seed. Such host plants are preferably plants of agronomic importance. It is contemplated that any element, or more than one element, of the host plant may be colonized with an endophyte to thus confer a host status to the plant. The initial inoculated element may additionally be different than the element to which the endophyte localizes. An endophyte may localize to different elements of the same plant in a spatial or temporal manner. For example, a seed may be inoculated with an endophyte, and upon germination, the endophyte may localize to root tissue.

The amount of endophyte that is used to inoculate a plant is preferably an amount effective to colonize a plant.

Any part of the plant may be inoculated with the endophyte of the present invention, including but not limited to a whole plant, seedling, meristematic tissue, ground tissue, vascular tissue, dermal tissue, seed, leaf, root, shoot, stem, flower, fruit, stolon, bulb, tuber, corm, kelkis, shoot, bud. According to a particular embodiment, the seed is inoculated with the endophyte of the present invention. For example, the endophyte of the present invention may be coated onto the surface of a seed. In another embodiment, the root may be inoculated with the endophyte of the present invention. In yet another embodiment, the plant may be inoculated by the endophyte of the present invention by foliar application.

In one embodiment, the plants (or parts thereof) are inoculated by direct contact.

In another embodiment, the plants (or parts thereof) are inoculated indirectly (e.g. via the soil, or via plant cultivation).

Methods of inoculating the plant or part thereof include, but are not limited to foliar inoculation, and/or soil inoculation and/or seed treatment and/or hydroponic application and/or drenching and/or fertigation and/or through irrigation systems.

Following inoculation the plant or part thereof (e.g. seed) is grown for at least one week, two weeks, three weeks, four weeks, five weeks, six weeks, seven weeks, eight weeks, nine weeks, ten weeks or more.

In one embodiment, the growing is effected under water limiting conditions or under abiotic stress conditions.

Following growing, the plant which has been inoculated may be selected and/or harvested.

According to another aspect of the present invention there is provided a plant or part thereof (e.g. seed) comprising an exogenous population of the endophytes of the present invention. In one embodiment, the plant is a cultivated plant as further described herein above. The endophytes are disposed on an exterior surface of or within the plant or part thereof (e.g. seed) in an amount effective to colonize the plant or part thereof (e.g. seed). The population is considered exogenous to the plant if that particular plant does not inherently contain the population of endophytic microbial entities.

As shown in the Examples section below, the endophytic populations described herein are capable of colonizing the host plant. In certain cases, the endophytic population can be applied to the plant, for example the plant seed, or by foliar application, and successful colonization can be confirmed by detecting the presence of the endophytic microbial population within the plant. For example, after applying the endophyte to the seeds, high titers of the endophyte may be detected in the roots and shoots of the plants that germinate from the seeds. In addition, significant quantities of the endophyte may be detected in the rhizosphere of the plants. Therefore, in one embodiment, the endophytic microbe population is disposed in an amount effective to colonize the plant. Colonization of the plant can be detected, for example, by detecting the presence of the endophytic microbe inside the plant. This can be accomplished by measuring the viability of the microbe after surface sterilization of the seed or the plant: endophytic colonization results in an internal localization of the microbe, rendering it resistant to conditions of surface sterilization. The presence and quantity of the microbe can also be established using other means known in the art, for example, immunofluorescence microscopy using microbe specific antibodies, or fluorescence in situ hybridization (see, for example, Amann et al. (2001) Current Opinion in Biotechnology 12:231-236, incorporated herein by reference in its entirety). Alternatively, specific nucleic acid probes recognizing conserved sequences from the endophytic bacterium can be employed to amplify a region, for example by quantitative PCR, and correlated to CFUs by means of a standard curve.

The endophytic populations described herein are capable of providing agronomic benefits to the host plant. As shown in the Examples section herein below, endophyte-inoculated plants display increased drought tolerance, abiotic stress tolerance, increased vigor, increased biomass (e.g., increased root or shoot biomass). Therefore, in one embodiment, the population is disposed on the surface or within a tissue of the seed or seedling in an amount effective to increase the biomass of the plant, or a part or tissue of the plant grown from the seed or seedling. The present invention contemplates a plurality of such seeds (e.g. 1000 seeds).

The increased biomass is useful in the production of commodity products derived from the plant. Such commodity products include an animal feed, a fish fodder, a cereal product, a processed human-food product, a sugar or an alcohol. Such products may be a fermentation product or a fermentable product, one such exemplary product is a biofuel. The increase in biomass can occur in a part of the plant (e.g., the root tissue, shoots, leaves, etc.), or can be an increase in overall biomass. Increased biomass production refers to at least about 10%, 20%, 30%, 40%, 50%, 60%, 70%, 80%, 90%, 100%, or greater than 100% when compared with a reference agricultural plant. Such increase in overall biomass can be under relatively stress-free conditions. In other cases, the increase in biomass can be in plants grown under any number of abiotic or biotic stresses, including drought stress, salt stress, heat stress, cold stress, low nutrient stress, nematode stress, insect herbivory stress, fungal pathogen stress, bacterial pathogen stress, and viral pathogen stress. In one particular embodiment, the endophytic microbial population is disposed in an amount effective to increase root biomass by at least 10%, for example, at least 20%, at least 30%, at least 40%, at least 50%, at least 60%, at least 75%, at least 100%, or more, when compared with a reference agricultural plant.

In another embodiment, the endophytic microbial population is disposed on the surface or within a tissue of the seed or seedling in an amount effective to increase the rate of seed germination when compared with a reference agricultural plant. For example, the increase in seed germination can be at least 2%, at least 3%, at least 4%, at least 5%, at least 6%, at least 7%, at least 8%, at least 9%, at least 10%, at least 15%, for example, at least 20%, at least 30%, at least 40%, at least 50%, at least 60%, at least 75%, at least 100%, or more, when compared with a reference agricultural plant.

In other cases, the endophytic microbe is disposed on the plant or part thereof (e.g. seed or seedling) in an amount effective to increase the average biomass of the fruit or cob from the resulting plant by at least 2%, at least 3%, at least 4%, at least 5%, at least 6%, at least 7%, at least 8%, at least 9%, at least 10%, at least 15%, for example, at least 20%, at least 30%, at least 40%, at least 50%, at least 75%, at least 100% or more, when compared with a reference agricultural plant.

In another embodiment, the present invention provides for a seed comprising an endophytic microbial population which is disposed on the surface or within a tissue of the seed or seedling in an amount effective to increase the height of the plant. For example, the endophytic microbial population is disposed in an amount effective to result in an increase in height of the agricultural plant such that is at least 10% greater, for example, at least 20% greater, at least 30% greater, at least 40% greater, at least 50% greater, at least 60% greater, at least 70% greater, at least 80% greater, at least 90% greater, at least 100% greater, at least 125% greater, at least 150% greater or more, when compared with a reference agricultural plant, the plant. Such increase in height can be under relatively stress-free conditions. In other cases, the increase in height can be in plants grown under any number of abiotic or biotic stresses, including drought stress, salt stress, heat stress, cold stress, low nutrient stress, nematode stress, insect herbivory stress, fungal pathogen stress, bacterial pathogen stress, and viral pathogen stress.

The host plants inoculated with the endophytic microbial population also show dramatic improvements in their ability to utilize water more efficiently. Water use efficiency is a parameter often correlated with drought tolerance. Water use efficiency (WUE) is a parameter often correlated with drought tolerance, and is the C02 assimilation rate per water transpired by the plant. An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

When soil water is depleted or if water is not available during periods of drought, crop yields are restricted. Plant water deficit develops if transpiration from leaves exceeds the supply of water from the roots. The available water supply is related to the amount of water held in the soil and the ability of the plant to reach that water with its root system. Transpiration of water from leaves is linked to the fixation of carbon dioxide by photosynthesis through the stomata. The two processes are positively correlated so that high carbon dioxide influx through photosynthesis is closely linked to water loss by transpiration. As water transpires from the leaf, leaf water potential is reduced and the stomata tend to close in a hydraulic process limiting the amount of photosynthesis. Since crop yield is dependent on the fixation of carbon dioxide in photosynthesis, water uptake and transpiration are contributing factors to crop yield. Plants which are able to use less water to fix the same amount of carbon dioxide or which are able to function normally at a lower water potential have the potential to conduct more photosynthesis and thereby to produce more biomass and economic yield in many agricultural systems. An increased water use efficiency of the plant relates in some cases to an increased fruit/kernel size or number.

Therefore, in one embodiment, the plants described herein exhibit an increased water use efficiency (WUE) when compared with a reference agricultural plant grown under the same conditions. For example, the plants comprising the endophytic microbial population (e.g. grown from the seeds comprising the endophytic microbial population) can have at least 5% higher WUE, for example, at least 10% higher, at least 20% higher, at least 30% higher, at least 40% higher, at least 50% higher, at least 60% higher, at least 70% higher, at least 80% higher, at least 90% higher, at least 100% higher WUE than a reference agricultural plant grown under the same conditions. Such an increase in WUE can occur under conditions without water deficit, or under conditions of water deficit, for example, when the soil water content is less than or equal to 60% of water saturated soil, for example, less than or equal to 50%, less than or equal to 40%, less than or equal to 30%, less than or equal to 20%, less than or equal to 10% of water saturated soil on a weight basis.

In a related embodiment, the plant comprising the endophytic microbial population can have at least 10% higher relative water content (RWC), for example, at least 20% higher, at least 30% higher, at least 40% higher, at least 50% higher, at least 60% higher, at least 70% higher, at least 80% higher, at least 90% higher, at least 100% higher RWC than a reference agricultural plant grown under the same conditions.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

1. General Procedures, DNA Extraction and Sequencing, and Sequence Analysis

Plant Material:

Plant species examined included Aegilops sharonensis (sharon goatgrass; AS), Triticum dicoccoides (wild emmer wheat; TD) and Triticum aestivum (bread wheat; TA). Plants were collected over the course of one year from natural habitats (AS and TD) and cultivated fields (TA) in Israel. Fresh plant samples were collected at the heading stage and spikelets were collected at late ripening. Each plant/spikelet was placed in an individual, numbered, paper bag. Fresh plants were transferred to the lab in a chilled cooler and stored at 4° C. for a maximum of 24 hours, before processing. Spikelets were kept in sealed paper bags under ambient conditions for up to 3 months prior to processing or propagation.

Production of Plants from Seeds (F1 Plants).

Unsterilized seeds were vernalized for 72 hours in darkness at 4° C. Seeds were further germinated for two days on a sterilized, wet filter paper under ambient conditions. Emerging seedlings were planted in sterile sea sand (sterilized twice by autoclave) and grown in a greenhouse at 22° C. for 3-4 weeks, until plants reached a height of 10-20 cm. A minimum of 4 progeny plants were propagated from each spikelet, to represent a single parental plant.

Isolation and Storage of Fungal Endophytes.

Endophytes were isolated from plant stems or seeds according to Schultz et al. (Schulz, Wanke, Draeger, & Aust, 1993). The leaves were removed and the stems were sectioned into 4-5 cm pieces prior to sterilization. Seeds were manually separated from each spikelet and the seed coat was removed. Tissues were surface sterilized by dipping in 70% ethanol for 30 seconds and then in 0.5% active chlorine for 2 minutes. Following sterilization, the tissue was washed twice with sterile distilled water and then sectioned using a sterile scalpel. Cuttings were placed on 10% potato dextrose agar medium (10% PDA) supplemented with 100 μg/ml ampicillin and 50 μg/ml chloramphenicol. The samples were incubated at 23° C. for 1-4 weeks until emergence of mycelia. Samples of mycelia were removed aseptically to fresh PDA plates, and the developing colonies were purified by repeated transfer of samples to fresh medium until a uniform and visually pure culture was obtained. For long-term preservation, mycelia and spores were collected into 20% glycerol in water (v/v) and stored at −80° C. In addition, cultures were produced on PDA slants in Eppendorf tubes and stored under sterile mineral oil at 4° C.

DNA Extraction, ITS Amplification and Sequencing.

Mycelia for DNA extraction were obtained from fresh axenic cultures. DNA was extracted using the Extract-N-Amp tissue polymerase chain reaction (PCR) kit (Sigma-Aldrich Corporation, Missouri, USA) as described by Kennedy et al. (Kennedy, Peay, & Bruns, 2009). For samples that did not yield adequate DNA by this procedure, DNA was extracted according to Cenis (1992) with 50 mg of lyophilized mycelium.

The fungal ribosomal intergenic spacer region 1 (ITS1), 5.8S and ITS2 regions were amplified using the fungal-specific ITS1 and ITS4 primers (Gardes & Bruns, 1993). PCR reactions were carried out in a volume of 40 μl using 1 U of DreamTaq green DNA polymerase (Thermo Scientific™ New_Hampshire, USA), 2 μl Dimethyl sulfoxide (DMSO), 10 pmol of each primer, 0.2 μM dNTP's and 1-2 μl of template DNA. Amplification was conducted using the following settings: an initial denaturation step of 94° C. for 3 min, 32 cycles that included [denaturation at 94° C. for 30 seconds, annealing at 52° C. for 45 seconds, polymerization at 72° C. for 45 seconds] and a final extension step of 10 min at 72° C. PCR products were analyzed by electrophoresis on 1% agarose followed by staining with ethidium bromide and visualization under UV light. Samples that PCR products produced a clear visible band were purified with ExoSAP-IT® (USB Corporation, Ohio, USA) sequenced by the Sanger method.

Sequence Processing and OTU Grouping.

Quality control of the raw sequence data was performed in two stages: first, the 5′ and 3′ ends were trimmed by removing bases ranking below Phred score of 27. Then, average quality was calculated for a moving window of 30 bases and sequences were trimmed accordingly using a 35 Phred score average threshold. Trimmed, good quality sequences of 350 bases or longer were curated. This process generated 601 (out of 741 input files) sequences with an average size of 491 bp.

Sequences were used to calculate unaligned distances between sequences was determined by a K-Mer algorithm (K=7). A draft of calculated distances was generated and sequences were assigned to groups using UPGMA algorithm based on <35% sequence dissimilarity. For each group, MUSCLE multiple alignment was performed and 95% of the non-nucleotide characters that are added by the algorithm were removed from the ends of each sequence (Edgar, 2004). By removing these characters prior to calculating distances, we were able to optimize the coverage to achieve better distance calculation within each group. The present inventors then calculated aligned distances in each group using Kimura-2d parameter in order to produce a distance matrix and the UPGMA method to build an accurate distance tree based upon calculated distances. This process enabled clustering of all the “good quality” sequences into OTUs based on 97% similarity at each node within each group.

Data Analysis.

OTU taxonomy was determined using classify.seqs command in MOTHUR (Version 1.34.1; Schloss et al., 2009) with the UNITE ITS database (version 7) as template (Kõljalg et al., 2013) Each OTU was classified up to the genus level. Where data indicated higher resolution of classification, inference was made to the best species or section within the genus. Chaol and Dominance D diversity indices were calculated using PAST 2.17c statistical analysis software.

Next-Generation ITS Amplicon Sequencing and Data Analysis.

ITS amplicon sequencing was performed for stem samples of T. dicoccoides and A. sharonensis. Six individual plants were collected from field populations at Almagor and Palmachim respectively. Plants were surface sterilized as described above and stems were cut into 3-5 mm sections. 200 mg of fresh tissue was lyophilized in 2 mL tubes containing two sterilized stainless-steel beads (4.8 mm diameter). Lyophilized material was then homogenized by bead beating for 2 min at 26 Hz using TissueLyser II (QIAGEN, Hilden, Germany). DNA was extracted with the GenEx™ Plant (plus!) kit (GeneAll Biotechnology Co., Seoul, Korea) according to the manufacturer instructions. Yield and quality of extracted DNA were determined spectrophotometrically using NanoDrop® ND-1000 (NanoDrop Technologies Inc., Wilmington, USA). Accordingly, DNA concentrations were adjusted to 50 ng/μL for further use in PCR steps. Fungal ITS1 was amplified using the primer set NSI1 (Martin & Rygiewicz, 2005) and ITS2 (White, Bruns, Lee, & Taylor, 1990). PCR was performed in 25 μL reaction volume using the KAPA HiFi HotStart ReadyMix® (KAPA Biosystems Inc., Massachusetts, USA), with 300 nM of each primer and 1 μL of template DNA. Reaction conditions were as follows: 95° C. for 2 min, followed by 28 cycles of [denaturation at 98° C. for 20 sec, annealing at 56° C. for 15 sec and elongation at 72° C. for 10 sec] and final 3 min elongation step at 72° C. Products of the PCR reaction were examined by agarose gel electrophoresis and 4 μL were used as template in a second PCR step, in which 5′ sequence tags were incorporated (common sequence 1 and 2, CS1 and CS2, (Moonsamy et al., 2013)). The reaction conditions and cycling conditions were as described above, but with five, instead of 28 cycles. Individual sample barcoding, library preparation for sequencing and library sequencing on Illumina MiSeq in 2×300 bp paired end format, were all performed at the DNA services (DNAS) facility, within the Research Resources Center (RRC) at the University of Illinois at Chicago (UIC).

Data obtained (733,000 paired end reads) was trimmed for low quality (Q30), sequences, barcodes and primers were removed, and sequences were further processed in MOTHUR version 1.34.1 (Schloss et al., 2009). Briefly, overlapping reads were merged using merge.contigs command and only completely assembled reads of 320-375 bases were utilized for further analysis. Omitting PhiX positive control (1.81%) and suspected chimeric (chimera.uchime, 5.3%) reads, 503,492 sequences were retained, and formed 24,119 unique (100% match) groups. The most abundant 200 unique sequence groups, representing the 86% of the reads, were used for generation of self reference database. Those sequences were aligned using MUSCLE multiple alignment algorithm and the alignment was manually inspected and corrected. All sequences were aligned to the generated self-reference and OTUs were defined at 97% sequence similarity. After random subsampling of the data to 10,000 reads per sample, the number of total detected culture-independent OTUs (ciOTUs), at the level of 97% similarity, was 282 (table S4). ciOTU taxonomy was determined up to the genus level using classify.seqs with the UNITE ITS database (version 7) as template. For ciOTUs of >0.5% relative abundance (RA), taxonomy was also examined by BLAST against the NR database of NCBI, excluding uncultured/environmental sequences, and taxonomy was inferred from the consensus of the first 10 top hits. In addition, the relatedness between cultivation independent and culture generated OTUs was examined. A representative sequence of the ciOTU was mapped against the culture collection set of ITS sequences using Lastz algorithm (Harris, 2007) implemented in the Galaxy{Blankenberg, 2001 #289) platform (www.//usegalaxy(dot)org/). Best hits were inspected manually by alignment in MEGA (version 6.06). Significant association between ciOTU and cultured OTUs was concluded if pairwise alignment was above 97% identical. Endophytic fungal communities of the two plant species were compared by non-metric multidimensional scaling analysis (NMDS) and analysis of similarities (ANOSIM) using R package VEGAN (version 2.3-0). Analysis of differential abundance of ciOTUs between AS and TD communities was conducted with R package edgeR (version 3.10.5). A negative binomial model was applied, and differential abundance was tested, based on quantile-adjusted conditional maximum likelihood method. Differential abundance was considered significant under the conditions that the difference in abundance between communities was above twofold and FDR-adjusted P value was <0.05.

2. Procedures for Characterization of Effect of Endophytes on Plant Growth

Plant Material.

Wheat seeds (Triticum aestivum cv Galil) were used in the present study. Before use, the seeds were surface-sterilized as described above. After sterilization, the seeds were soaked in sterile distilled water and placed in a Petri dish for vernalization at 5° C. for 24 h. Then the seeds were allowed to germinate for 48 hours in a growth chamber.

Fungi.

Isolates 14005 and 13237 were used in this study. Isolate #14005 (OTU 43) was obtained from stems of F1 plants that were produced from seeds of T. dicoccoides that were collected at Zefat site (LID013). Seven isolates comprising this OTU were detected solely in F1 T. dicoccoides plants propagated from seeds collected at Zefat and Amiad Junction sites (LID013 and LIDO11). Isolate #13237 (OTU 71) was obtained from stems of F1 plants that were produced from seeds of A. sharonensis that was collected at Palmachim (LID007) site. Two additional isolates of this OUT were detected in T. aestivum and A. sharonensis F1 plants collected at Naaman and Palmachim sites (LID038 and LID007).

DNA, PCR, Sequencing.

Fungal DNA was isolated as described above. For determination of taxonomic identity of isolates 13237 and 14005, the following genes were amplified and sequenced: elongation factor 1α (EF-1α), second large subunit of RNA polymerase II (RPB2), and ITS1,2. These genomic regions were amplified using primers that have been previously described in Sung et al. (2007) and Berbee et al. (1999). According to these sequences, the most closely related species to isolate 13237 is Acremonium sclerotigenum (Accessions KC999024, KC998988 and KC987166), and the most closely related species to isolate 14005 is Sarocladium implicatum (Accessions KT878359 and GU189520).

Production of Spores:

Mycelia were obtained from one week old PDA cultures and used to inoculate flasks containing 150 mL of Potato Dextrose Broth (PDB) medium. The cultures were incubated for five days in a growth chamber under condition of 25° C., continuous light, and agitation at 180 rpm. Following incubation, spores were collected by filtration of the cultures through two layers of Miracloth (Calbiochem), the spores were resuspended in water, counted and diluted in water to a final concentration 10⁶ conidia/mL.

Inoculation of Wheat Seedlings.

Seeds were Germinated for Two Days as Described above and seedlings with similar root and shoot size were selected. The roots of the selected seedlings were soaked for one hour in a spore suspension. Control plants were treated with sterile water. After soaking in the spore suspension the plants were placed on a sterile filter paper for 15 minutes and then planted in soil.

Plant Growth Under Salinity Treatment.

Seedlings were planted in 1 L plastic pots containing thoroughly washed sterile sand. The plants were maintained in a greenhouse under the conditions that were described above. Each treatment included five pots with four seeds per pot. Plants were watered with a half-strength Hoagland nutrient solution every second day. Salt treatment was applied one week after planting by adding 0.1M or 0.2M NaCl to the nutrient solution. The plants were harvested and analyzed two weeks following the start of salt treatment.

Drought Treatment.

Seedlings were planted in 0.5 L plastic pots containing 400 g of sterile loam soil. The plants were maintained in a greenhouse under the conditions that were described above. Each treatment included five pots with four seeds per pot. Plants were watered with a half-strength Hoagland nutrient solution every second day. Drought (or limitation) treatment was applied by quitting irrigation of plants one week after planting. The plants were harvested ten days from the last watering, at which time the commercial wheat without endophytes reached a wilting point.

Physiological Parameters.

The following parameters were measured to evaluate effect of treatments on the plants: shoot and roots length, shoot and root biomass, root architecture, leaves width, and chlorophyll content. After harvest, the roots were washed thoroughly, dried and then photographed to obtain images of the root architecture. The length of the single longest root was used as a measure of root length. The height of the plant, as measured from the soil to the top leaf, was used as a measure of shoot length. The fresh weight of all above ground parts was used as a measure of shoot biomass and the fresh weight of all underground parts was used as a measure of root biomass. The width of the second blade was measured with ImageJ software as previously described (Juneau and Tarasoff, 2012) and used as measure of leaf width. Chlorophyll levels were measured using a chlorophyll meter CCM-200 plus (Opti-science). Three measurements were taken per leaf with 10 plants per treatment. The three CCI units (Chlorophyll Concentration Index) readings taken on one leaf were averaged to represent one observation. The results were obtained as CCI values (dimensionless).

Results

1. Endophytes Diversity in the Three Plant Species

Occurrence of Fungal Endophytes in Wheat and the Wild Species.

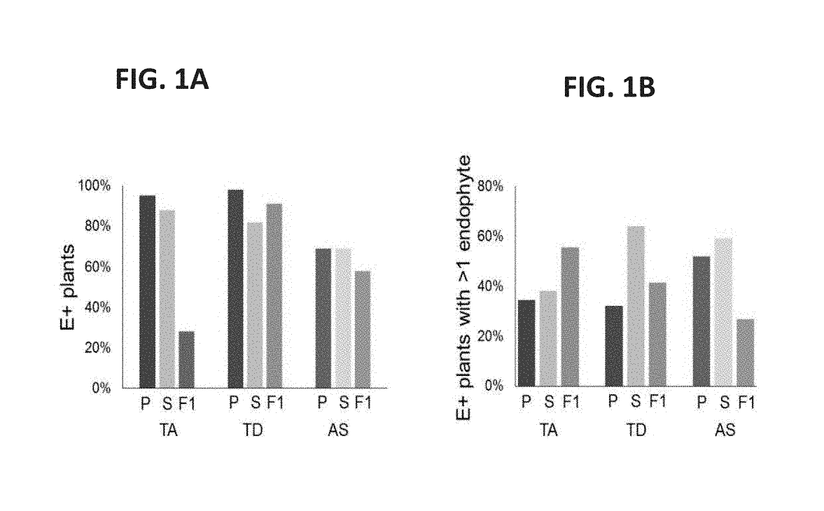

The occurrence of culturable species of endophytic fungi in stems and seeds of TA, TD and AS was determined. A total of 233 stem samples (P) and 100 seed samples (S) were evaluated. In addition, 90 samples were analyzed from stems of plants that were produced in a sterile soil in the greenhouse from field collected seeds (F1). Incidence of endophytes (E+) was calculated for seeds and stems of each species separately, as the percentage of samples that yielded at least one endophyte per sample (FIG. 1A; Table 1A). Incidence of E+ plants was similar in field collected stem samples of TA and TD (95% and 98%, respectively) and was significantly lower in AS (69%, χ² test, P<0.001). Conversely, seeds of TA and TD contained lower incidence of endophytes compared to respective stems (χ² test, P<0.01), whereas in AS seeds and stems had a similar incidence of endophytes. The incidence of endophytes in the F1 samples was significantly lower (χ² test, P<0.05) for all three species compared with the incidence in corresponding parental samples.

TABLE 1A
Incidence of endophytic fungi (E+) and multiple endophytic fungi
(E = n) in T. aestivum (TA), T. dicoccoides (TD) and A. sharonensis
(AS) in plant stems (P), seeds(S) or F1 plant stems (F1).

| Sample | Plant | No. | E+ | | | | | |
|---|---|---|---|---|---|---|---|---|
| type | specie | plants | Total | % | E = 1 | E = 2 | E = 3 | E = 4 |
| P | TA | 74 | 70 | 95 | 40 | 15 | 6 | 0 |
| P | TD | 108 | 106 | 98 | 61 | 26 | 2 | 1 |
| P | AS | 51 | 35 | 69 | 13 | 5 | 8 | 1 |
| S | TA | 34 | 30 | 88 | 18 | 8 | 3 | 0 |
| S | TD | 34 | 28 | 82 | 9 | 13 | 3 | 0 |
| S | AS | 32 | 22 | 69 | 9 | 8 | 4 | 1 |
| F1 | TA | 32 | 9 | 28 | 4 | 4 | 1 | 0 |
| F1 | TD | 32 | 29 | 91 | 17 | 10 | 2 | 0 |
| F1 | AS | 26 | 15 | 58 | 11 | 3 | 1 | 0 |

In total, 686 cultures were produced and the ITS sequence was obtained from 514 of them (78%). Categorization into OTUs at 97% sequence similarity resulted in 67 discrete groups. The number of samples with more than a single OTU varied between plant species and plant parts, ranging between 33% to 64% of the samples (FIG. 1B, Table 1A).

Structure of Endophytic Fungal Community.

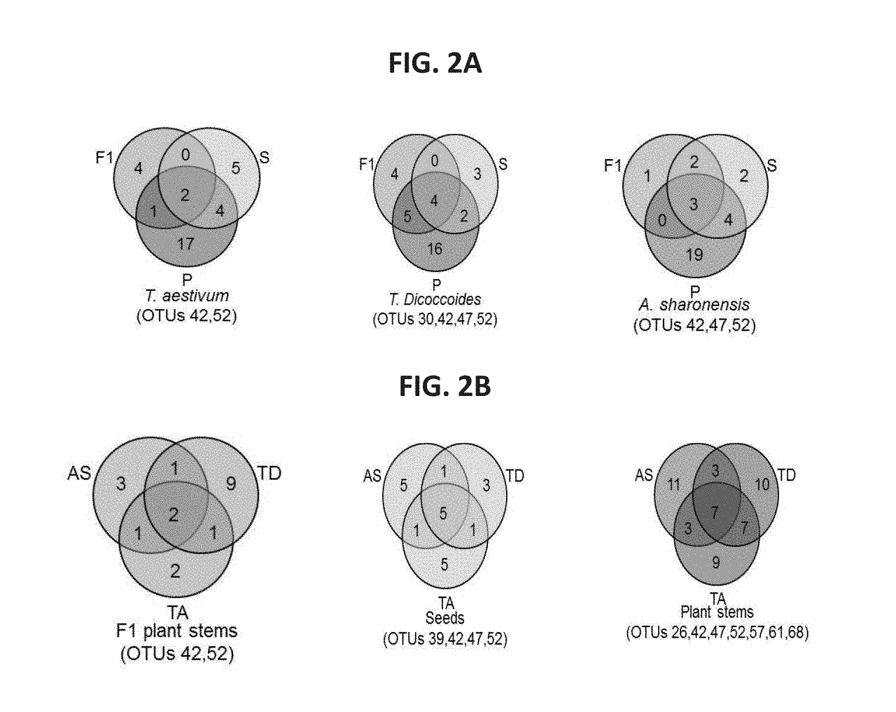

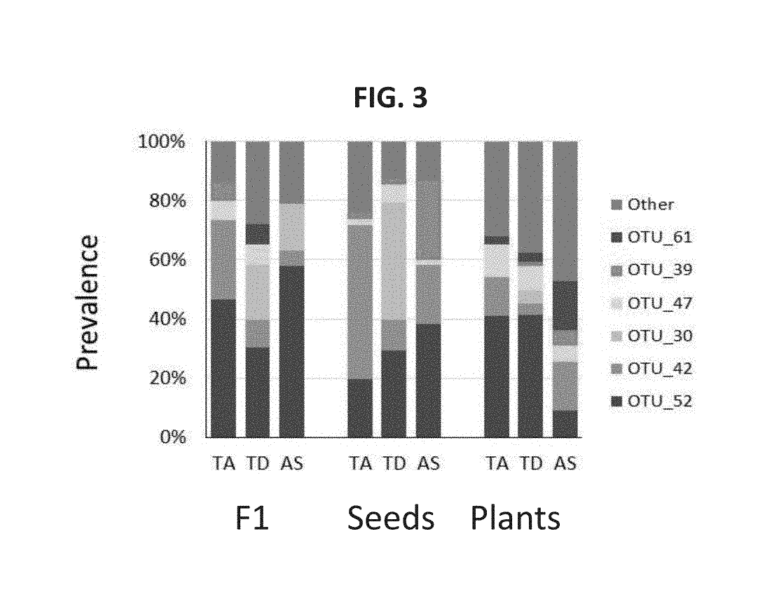

For all plant species, OTU richness was more than twice in stems compared to seed or F1 plant samples, and in total more than half of the OTUs (36 out of 67) were only detected in stems (FIG. 2B). In addition, a high proportion of the OTUs in each combination of plant species and sample type (44%-77%) appeared as singletons or doubletons (Table 1B). However, a clear set of OTUs were highly prevalent in all plant species and all sample types. Of these, the most prevalent OTUs were OTU_52 and OTU_42, which were found in 35% and 17% of the samples, respectively, and OTU_47, which was also detected in all plant (FIGS. 2A-B). Altogether, the top six most prevalent OTUs accounted for 70% of the total isolates collection, and between 53% and 87% of the isolates for each of the examined samples (FIG. 3).

TABLE 1B
Diversity of T. aestivum (TA), T. dicoccoides (TD) and A. sharonensis
(AS) endophytic communities in plant stems (P), seeds(S) or F1 plant stems (F1).
Chao1 richness estimator and community dominance index (D) are presented.

| | | No. of | No. of | | | | |
|---|---|---|---|---|---|---|---|
| ^{a}Sample | ^{b}Plant | isolates | OTUs | Singletons | Doubletons | Chao1 | Dominance |
| P | TA | 100 | 26 | 14 | 6 | 39 | 0.20 |
| P | TD | 133 | 27 | 15 | 4 | 48 | 0.20 |
| P | AS | 55 | 24 | 16 | 2 | 64 | 0.09 |
| S | TA | 46 | 11 | 6 | 2 | 16 | 0.32 |
| S | TD | 48 | 9 | 4 | 0 | 15 | 0.26 |
| S | AS | 55 | 11 | 8 | 0 | 39 | 0.26 |
| F1 | TA | 15 | 6 | 4 | 0 | 12 | 0.31 |
| F1 | TD | 43 | 13 | 6 | 1 | 20.5 | 0.16 |
| F1 | AS | 19 | 7 | 5 | 0 | 17 | 0.37 |

^{a}P—fresh plants, S—seeds, F1—seeds produced from plants in greenhouse
^{b}TA—Triticum aeastivum, TD—Triticum dicoccoides, AS—Aegilops sharonensis

Composition of Endophytic Fungal Community.

The majority of endophytes isolated in this work belong to the phylum Ascomycota, and are divided between five classes and seventeen orders. Only three OTUs (out of a total of 67) are in the phylum Basidiomycota and each belongs to a different order (Table 2).

TABLE 2
Endophytic taxonomy overview. Number
of OTUs in each taxa in parenthesis.

| Phylum | Class | Order |
|---|---|---|
| Ascomycota(64) | Dothideomycetes (33) | Pleosporales (29) |
| | | Botryosphaeriales (1) |
| | | Capnodiales (1) |
| | | Dothideales (1) |
| | | Unassigned (1) |
| | Sordariomycetes (22) | Hypocreales (7) |
| | | Sordariales (6) |
| | | Diaporthales (3) |
| | | Unassigned (2) |
| | | Trichosphaeriales (1) |
| | | Xylariales (3) |
| | Eurotiomycetes (7) | Eurotiales (7) |
| | Leotiomycetes (1) | Helotiales (1) |
| | Pezizomycetes (1) | Unassigned (1) |
| Basidiomycota (3) | Agaricostilbomycetes (1) | Agaricostilbales (1) |
| | Tremellomycetes (1) | Holtermanniales (1) |
| | Ustilaginomycetes (1) | Ustilaginales (1) |

Pleosporaceae, and particularly Alternaria spp. were the most commonly detected OTUs, occurring in all hosts and sample types. OTUs belonging to the Alternaria complex were grouped based on classification to sections proposed by Woundenberg et al. (2013). The Alternaria species were divided between three main subgroups: A. section Infectoria, A. sec. Alternata, and A. sec. chalastospora. Alternaria spp. of the section Infectoria (OTU_48-OTU_56) were found in all hosts and sample types. Sec. Infectoria was the dominant group of endophytes in field samples of TA and TD, comprising >40% of all fungal endophytes in stems of both species. In AS stems, the prevalence of sec. Infecoria was much lower than in stems of TA or TD (9.1%), while in seeds or F1 plants it was more prevalent in AS compared to the other two species. Alternaria spp. of section Alternata (OTU_40-OTU_42), were also prevalent in all hosts and sample types, and in particular in seeds of TA comprising 52% of isolates.

Other genera found as endophytes in most samples included Cladosporium (Capnodiales), which was present in all samples except for stems of AS F1 plants, Stemphylium (Pleosporales), which was highly prevalent in seeds of AS. In addition, Aspergillus, was found in all species but was restricted to stems. Chaetomium, which was found in samples of stems that were collected from field in all species, had high prevalence in AS (19.6%) and was also detected, but at low prevalence, in AS seeds and in F1 stems of TD.

Culture-Independent Assessment of Diversity.

In order to examine the relatedness between cultivated endophytic fungal diversity and actual diversity of endophytes, an amplicon mass-sequencing strategy was employed. DNA was extracted from 12 field collected stem samples of AS and TD (6 samples each) and partial ITS sequence (ITS1) was amplified and sequenced. The endophytic community of each individual plant sample was represented by 10,000 sequences (120,000 in total). Analysis of these amplicon sequences yielded a total of 282 culture independent OTUs (ciOTUs) at 97% sequence similarity. Of those, 50% were singletons or doubletons (relative abundance ≤0.02%; Table 3). The actual and estimated (Chaol) ciOTU richness per plant were similar between AS and TD (ANOVA, P>0.05). Likewise, average dominance values were statistically similar (Table 3). RA≥0.5% was considered a robust threshold for occurrence and only few ciOTUs per plant have reached this level (4-10 ciOTUs per plant in AS and 6-10 ciOTUs per plant in TD), in total 32 ciOTUs. In comparison, field plant stem samples of AS and TD yielded 40 unique cultivated OTUs that were isolated from a total of 159 plants. In addition, it was important to examine the probability of co-habitation of the plant by multiple endophytes. Considering only ciOTUs with high RA (≥10%), all but one AS plant harbored two and up to four dominant endophytic populations. These rates of E+plants with more than one endophyte was significantly higher than expected by cultivation method for TD (100% vs. 32%; χ²=11.15, P<0.001), but not for AS (83% vs. 52%; χ²=1.99, P>0.05).

TABLE 3
Numbers and diversity parameters of endophytic fungal communities of TD and AS field collected stems.
OTUs were defined at 3% sequence similarity. OTU richness was estimated by calculation of Chao1 index.
Dominance index represents the distribution of relative abundance among OTUs within each plant.

| | A. sharonensis | | | | | | T. dicoccoides | | | | | | |
|---|---|---|---|---|---|---|---|---|---|---|---|---|---|
| | ITS_P0 | ITS_P0 | ITS_P0 | ITS_P0 | ITS_P0 | ITS_P0 | ITS_P0 | ITS_P0 | ITS_P0 | ITS_P0 | ITS_P0 | ITS_P0 | |
| | 659 | 660 | 661 | 662 | 663 | 664 | 665 | 666 | 667 | 669 | 701 | 702 | Total |
| No. of OTUs | 37 | 29 | 90 | 79 | 49 | 50 | 85 | 74 | 67 | 86 | 84 | 45 | 282 |
| Singeltons | 19 | 11 | 41 | 35 | 24 | 17 | 26 | 30 | 25 | 31 | 34 | 20 | 112 |
| Doubltons | 5 | 7 | 27 | 18 | 12 | 16 | 21 | 18 | 13 | 18 | 19 | 14 | 67 |
| >0.5%* | 5 | 4 | 10 | 8 | 6 | 7 | 8 | 10 | 6 | 10 | 7 | 6 | 48 |
| >10%* | 1 | 2 | 3 | 3 | 3 | 2 | 3 | 3 | 2 | 2 | 4 | 4 | 11 |
| Chao1 | 80 | 43 | 193 | 198 | 84 | 84 | 118 | 118 | 92 | 111 | 140 | 108 | 483 |
| Dominance_D | 0.91 | 0.43 | 0.30 | 0.28 | 0.29 | 0.46 | 0.24 | 0.38 | 0.58 | 0.41 | 0.29 | 0.25 | 0.16 |

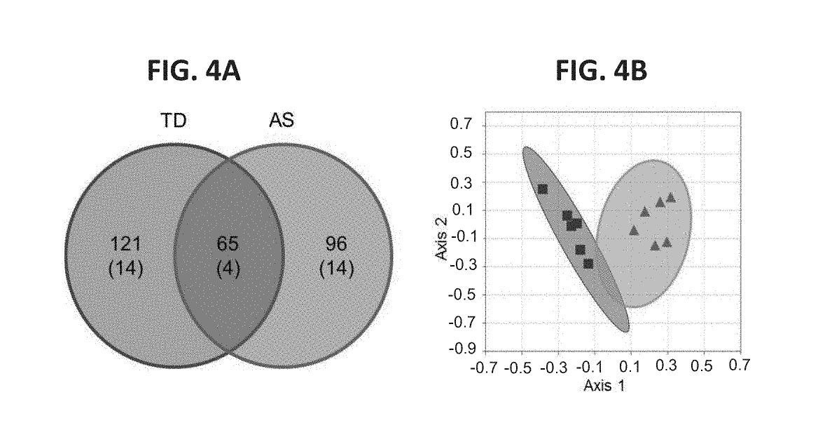

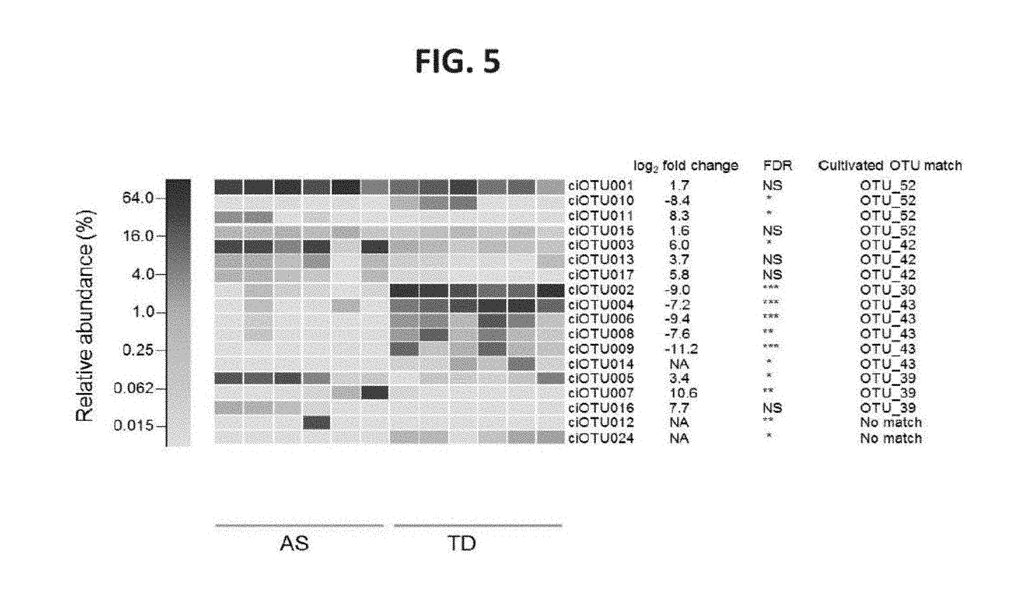

Similar to cultivation method results (FIG. 2B), the majority of ciOTUs detected were unique to the plant species (FIG. 4A). Only 23% of the ciOTUs, were detected in both AS and TD and, if considering only ciOTUs with RA>0.5%, this rate dropped by half. Dissimilarity between AS and TD communities was further examined by non-metric multidimensional scaling analysis (NMDS) using a Bray-Curtis distance matrix, and by analysis of similarities (ANOSIM) test. Both NMDS and ANOSIM (R=0.87, P<0.005) results supported significant disparity between the composition of AS and TD communities (FIG. 4B). In order to identify ciOTUs which significantly differ in abundance between AS and TD, the exact test based on the quantile-adjusted conditional maximum likelihood was applied and adjusted for false discovery. In total, 35 ciOTUs showed significant differences (FDR adjusted P<0.05). Those included all but two of the dominant (RA>10%) and/or prevalent (detected at >0.5% level in more than one plant) ciOTUs identified (FIG. 5). Interestingly, the number of ciOTUs with significant higher abundance in AS was only 5 compared to 30 ciOTUs favoring TD.

Taxonomic identification of the ciOTUs, carried out using the same method as for sequences obtained from the culture collection were much less robust of the amplicon sequences at the genus level. The present inventors therefore inferred higher resolution taxonomy based on BLAST search of NCBI database, and additionally used mapping strategy in order to explore the relatedness between isolate collection and ciOTUs (FIG. 5). Based on these analyses, the diversity captured by the amplicon sequencing approach represented for the most part a subset of the isolate culture collection obtained from AS and TD samples. Among the 32 ciOTUs with RA>0.5% at least in one plant, 26 matched a cultivated OTU (sequence similarity>97%), which comprised 94% and 96% of the total sequences for AS and TD respectively (FIG. 5). Alternaria was again found to be the dominant group of endophytes. Alternaria spp. sec Infectoria was again the most abundant and prevalent group. Alternaria sec. Chalastospora was found exclusively in TD. Stemphylium spp. which were isolated mainly from AS were dominant and prevalent ciOTUs in AS. Remarkable was the abundance of A. sec. Alternata in stems of TD (FIG. 5). These populations matched cultivated OTU_43 which was isolated only from TD F1 plants, but was not detected in field samples by cultivation. In contrast, some key genera isolated from TD or AS stems, namely Cladosporium, Chaetomium, Aspergillus and Phaeosphaeria, were not represented in prevalent ciOTUs. Among ciOTUs with RA>0.5% that had no match in the culture collection two were identified as Ustilaginaceae (Basidiomycota), two were Ophiocordycipitaceae, one was Cordycipitaceae and one Penicillium.

Sequences for the 13237 isolate are set forth in SEQ ID NO: 1 (ITS), SEQ ID NO: 2 (LSU), SEQ ID NO: 3 (EF1), SEQ ID NO: 4 (RBP-2) and SEQ ID NO: 5 (GPD).

2. Sequences for the 14005 isolate are set forth in SEQ ID NO: 6 (ITS), SEQ ID NO: 7 (LSU), SEQ ID NO: 8 (EF1), SEQ ID NO: 9 (RBP-2) and SEQ ID NO: 10 (B-tubulin).

Effect of the Two New Endophytes on Plants

Wheat plants were inoculated by dipping roots of 2-day old seedling in a spore suspension described in the methods. The plants were grown as described and the presence of the endophytes in the plants was verified by PCR with species-specific primer before harvesting and analysis of the plants, using DNA extracted therefrom.

The following primer sequences were used:

| Isolate | Gene | Sequences | |
|---|---|---|---|
| 13237 | RPB2 | Forward | 5′ GTGCGTCGTTGGGTTAGTCT 3′ |
| | | primer: | (SEQ ID NO: 11) |
| | | Reverse | 5′ CACCCAGCGAACCTCTCTAC 3′ |
| | | primer: | (SEQ ID NO: 12) |
| 14005 | EF | Forward | 5′ GTTCGAGGCTGGTATCTCCA 3′ |
| | | primer: | (SEQ ID NO: 13) |
| | | Reverse | 5′ GAGGACGATGACCTGAGCAT 3′ |
| | | primer: | (SEQ ID NO: 14) |

Normal and Salt Stress Conditions.

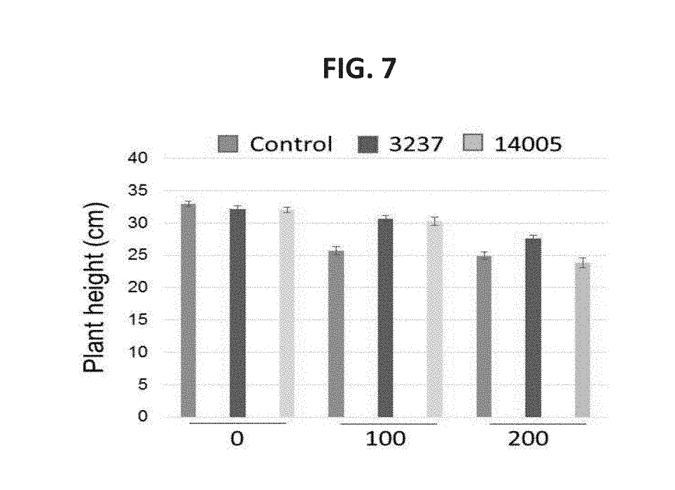

Salt treatment was applied one week after planting as described in methods. Plants were harvested three weeks after planting (three weeks after application of salt treatment). Overall, the endophyte-containing plants showed better performance under both normal and stress conditions as compared to the control plants without the endophytes. The overall appearance of endophyte-containing plants was dramatically improved compared with control plants, with elongated and greener shoots, and much more developed roots (FIG. 6). The overall better appearance of the endophyte-containing plants was reflected in the growth parameters, which were all higher in the endophyte-containing plants compared with control plants (FIGS. 7-12). Both endophytes had a similar positive effect on growth of plants under normal conditions, although the magnitude of the differences was lower than in salt stress conditions. Generally, the two endophytes had similar effects, with minor differences.

Drought Conditions.

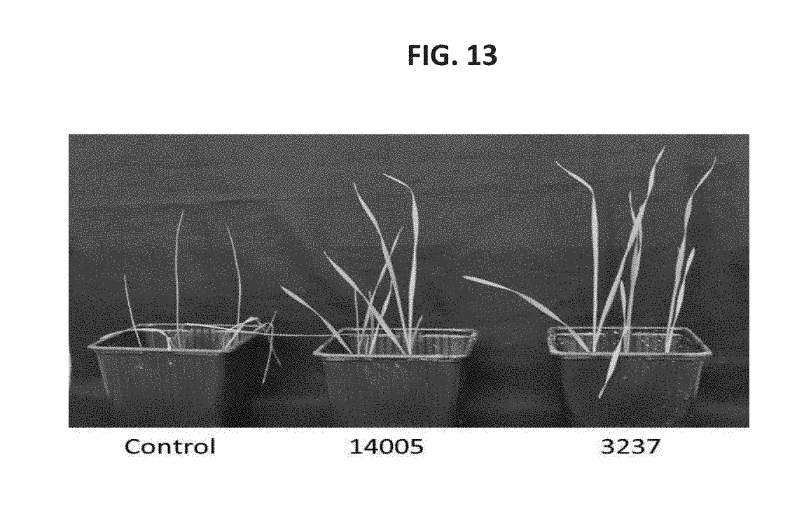

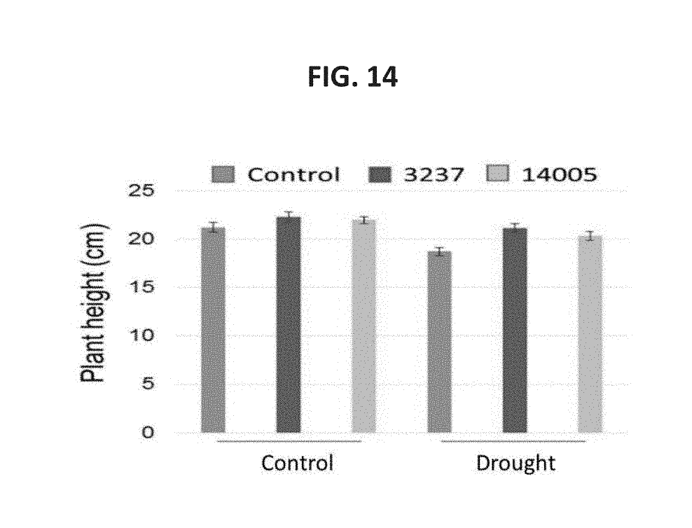

Drought treatment was applied as described in methods. Plants were harvested ten days after water supply was stopped, at which time the control plants reached wilting point. Overall, the endophyte-containing plants developed better and sustained longer under water limiting conditions (FIG. 13). Between the two endophytes, plants that were inoculated with isolate 13237 performed better than plants that were inoculated with isolate 14005. The overall better appearance of the endophyte-containing plants was reflected in the growth parameters, which were higher in the endophyte-containing plants compared with control plants (FIGS. 14-19).

### Example 2

### Additional Results Confirming Positive Effects on Wheat Growth

Enhanced Growth in Large Containers:

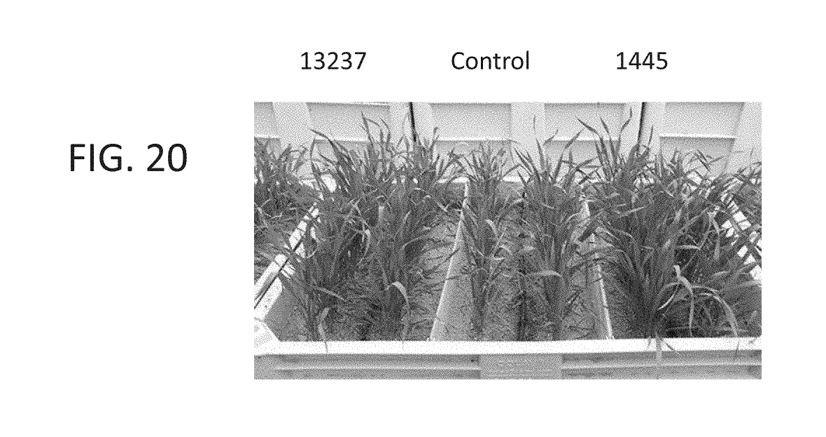

wheat seeds were inoculated with spores of either Acremonium (isolate 14005) or Serocladium (isolate 13237), seeds were planted in sand in large containers (80 cm×70 cm×60 cm). The plants were grown with irrigation and fertilization. Photographs were taken after 45 days (FIG. 20).

Enhanced Biomass and Number of Tillers in Field Plots:

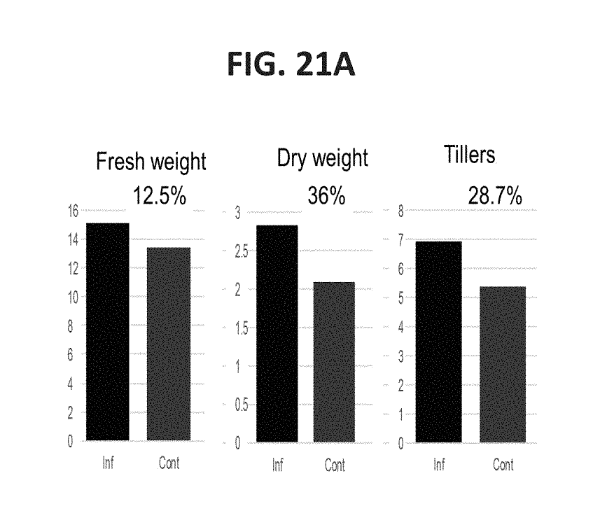

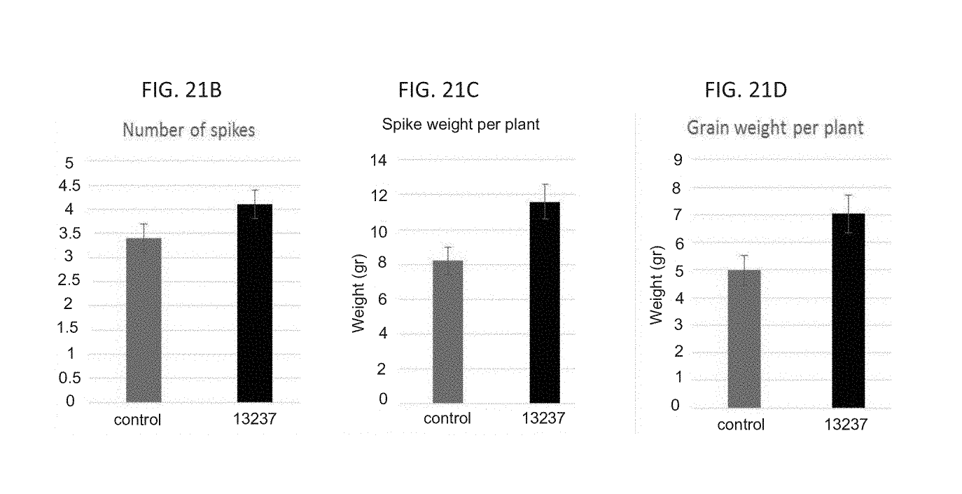

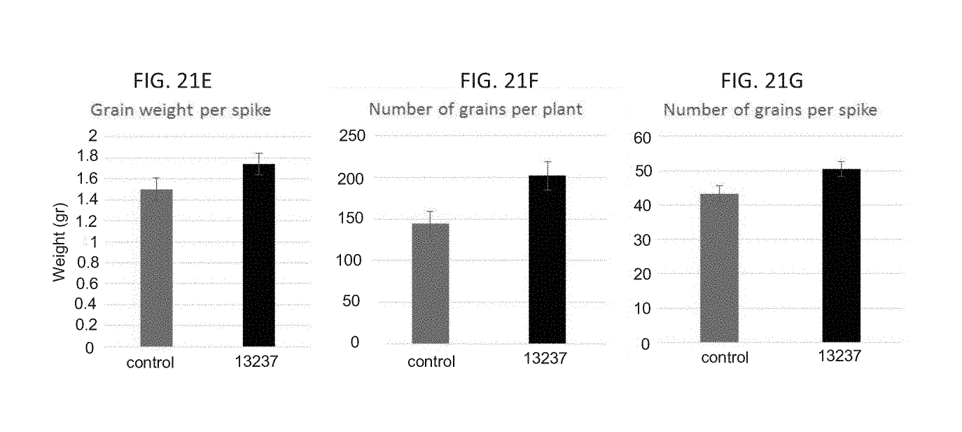

Wheat seeds were inoculated with Acremonium spores, the seeds were sawn using a commercial drill in field plots. Plants were grown until milk stage (60 days) and then samples were taken for evaluation of number of tillers, and shoot fresh and dry biomass (FIG. 21A). FIGS. 21B-G show the summary of yield data from samples taken at maturation.

Enhanced Number of Tillers in Greenhouse:

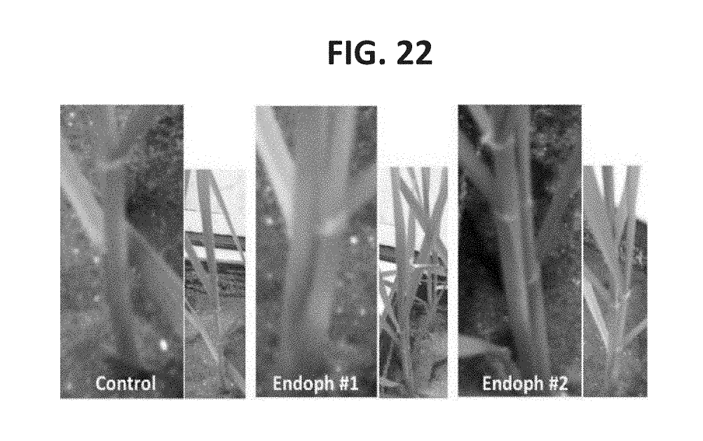

Wheat seeds were inoculated with spores of either Acremonium or Serocladium, planted in pots and maintained in greenhouse with optimal water and fertilization. After 45 days, plants treated with the endophytes developed two tillers compared with a single tiller in control plants (FIG. 22).

### Example 3

### Additional Results Confirming Positive Effects on Wheat Germination

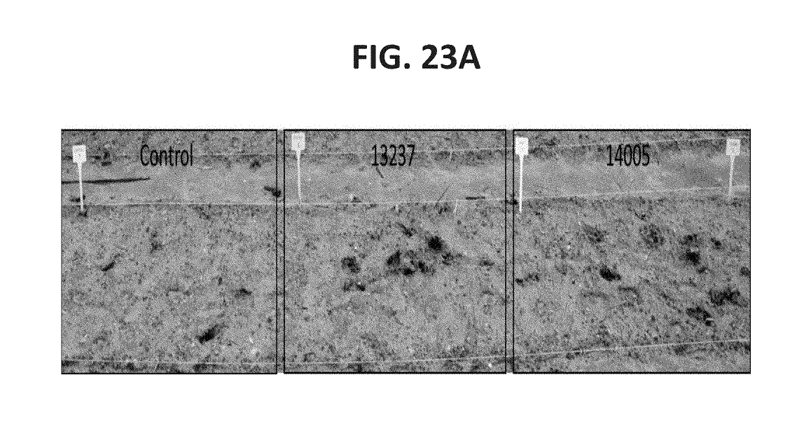

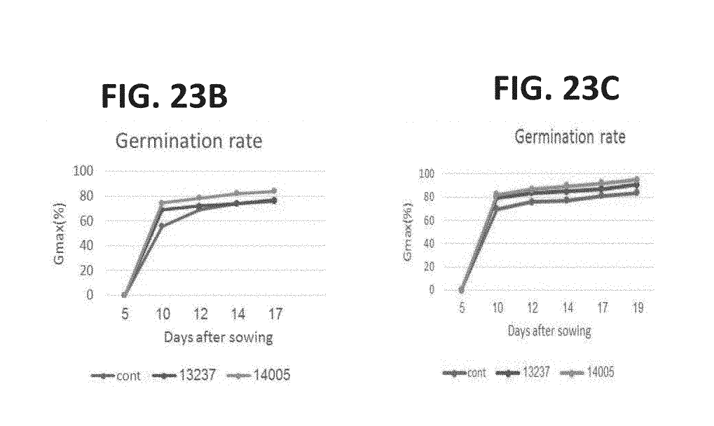

Wheat seeds were treated with spores of either Acremoniumor Serocladium and planted in small field plots. Seeds treated with endophytes showed earlier and more uniform germination (FIG. 23A). The data of relating to these photographs is presented in FIGS. 23B-C.

### Example 4

### Analysis of Stress-Related Metabolites

Materials and Methods

Proline Determination:

Root samples (0.5 g) from each group were homogenized in 3% (w/v) sulphosalycylic acid and the homogenates were filtered through filter paper. After addition of acid ninhydrin and glacial acetic acid, the resulting mixture was heated at 100° C. for 1 h in a water bath. The reaction was stopped by transferring of the samples to ice. The mixture was extracted with toluene, the toluene fraction was aspired from the liquid phase and the absorbance at 520 nm was recorded using a spectrophotometer. Proline concentration was determined using calibration curve and expressed as μmol proline g⁻¹ FW.

Lipid Peroxidation:

Lipid peroxidation was determined by measuring malondialdehyde (MDA) formation using the thiobarbituric acid method. For MDA extraction, 0.5 g of root samples was homogenized with 2.5 mL of 0.1% trichloroacetic acid (TCA). The homogenate was centrifuged for 10 min at 10,000×g. For every 1 mL of the aliquot, 4 mL of 20% TCA containing 0.5% thiobarbituric acid (TBA) was added. The mixture was heated at 95° C. for 30 min and then cooled quickly on ice. Afterwards, the mixture was centrifuged for 15 min at 10,000×g and the absorbance of the supernatant was measured at 532 nm. Measurements were corrected for unspecific turbidity by subtracting the absorbance at 600 nm. The concentration of MDA was calculated using an extinction coefficient of 155 mM⁻¹ cm⁻¹.

Chromatographic Analysis.

For hormone and phenolic compound analysis, fresh material was frozen in liquid nitrogen. Dry tissue (0.05 g) was immediately homogenized in 1 ml of ultrapure water and a mixture of internal standards was added before the extraction (100 ng of [2H6]-ABA, 100 ng of prostaglandin B1, dihydrojasmonic acid, and propylparaben. The extraction and experimental procedures were performed as described by Flors et al XXX please provide reference. After extraction, a 20-μl aliquot was directly injected into the UPLC system. Analyses were carried out using a Waters AQUITY UPLC system (Milford, Mass., U.S.A.) with nucleosil ODS reversed-phase column (100 by 2 mm i.d.; 5 μm) (Scharlab, Barcelona, Spain). The chromatographic system was interfaced to a Quatro LC (quadrupole-hexapolequadrupole) mass spectrometer (Micromass, Manchester, U.K.).

Results

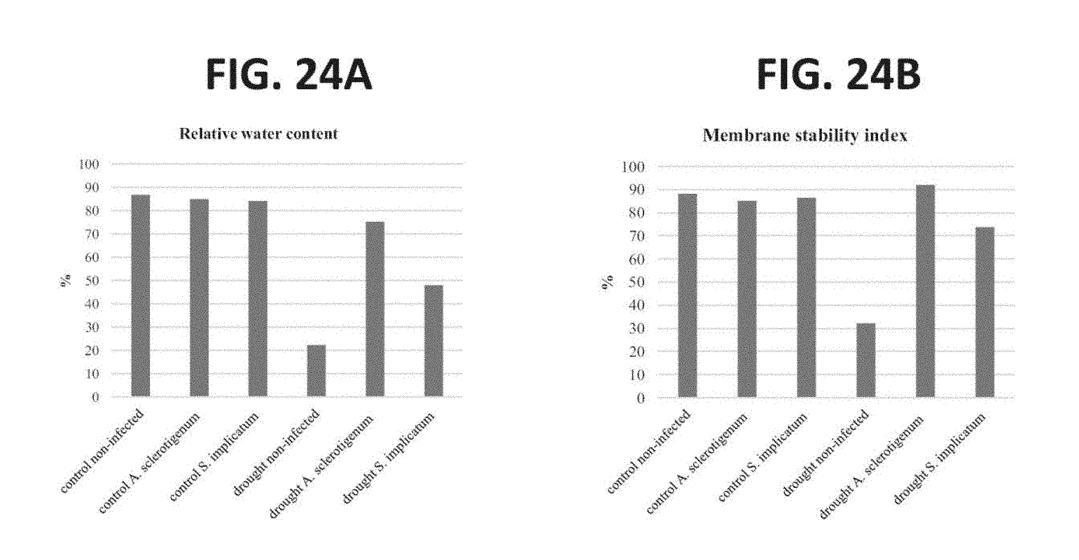

The relative water content of the inoculated plants once the un-inoculated controls reached the wilting point was three-fold higher in plants inoculated with A. sclerotigenum and two-fold higher in plants inoculated with S. implicatum. Non-stressed controls did not show significant differences in physiological performance between inoculated and non-inoculated plants. Severe water stress reduced relative water content in non-inoculated plants to 22%, whereas relative water content showed by inoculated plants was 75% for Acremonium inoculated plants and 50% for Sarocladium inoculated plants. The leaf relative water content of control plants did not differ between inoculated and non-inoculated (FIG. 24A).

Membrane stability index (msi) is the estimation of membrane dysfunction under stress by measuring cellular electrolyte leakage from plant tissue. The results show that the membrane stability index of non-inoculated plants under severe stress is below 35% whereas plants inoculated with Sarocladium showed values near 70%. Stressed plans inoculated with Acremonium did not show any significant difference in the MSI compared with the control plants, maintaining values higher than 90% (FIG. 24B).

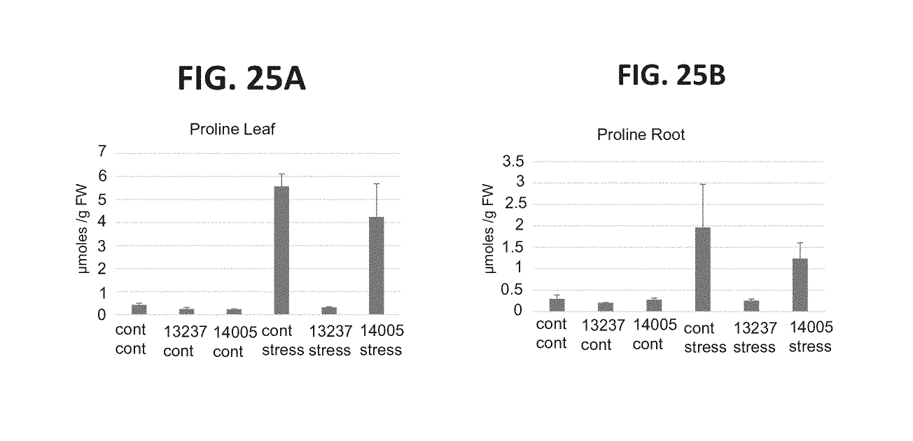

Proline accumulation is a common physiological response in plants exposed to abiotic stresses. The present results showed that plants inoculated with isolate 13237 and subjected to water stress showed similar levels of proline to control plants. However, a strong accumulation of this amino acid was observed in non-inoculated, stressed plants and 14005 stressed plants (FIGS. 25A-B).

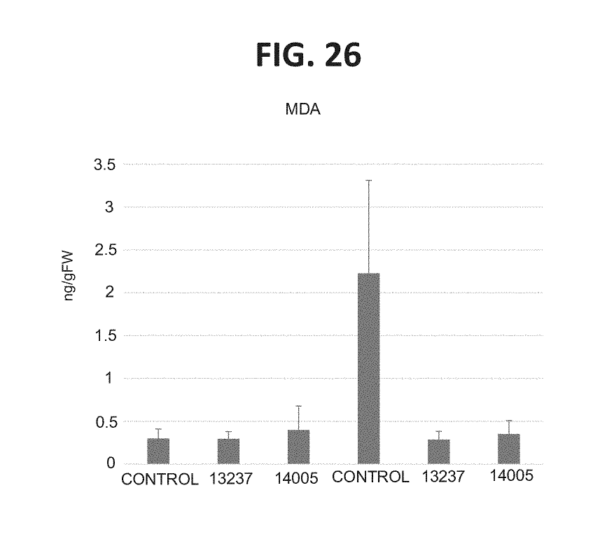

Lipid peroxidation estimated as MDA content was analyzed. MDA content increased with severe drought treatment. Non-inoculated stressed plants displayed 10 times more MDA content under severe drought treatments than well-watered plants. On the other hand, no significant differences were observed between inoculated and stressed plants with the well-watered plants (FIG. 26).

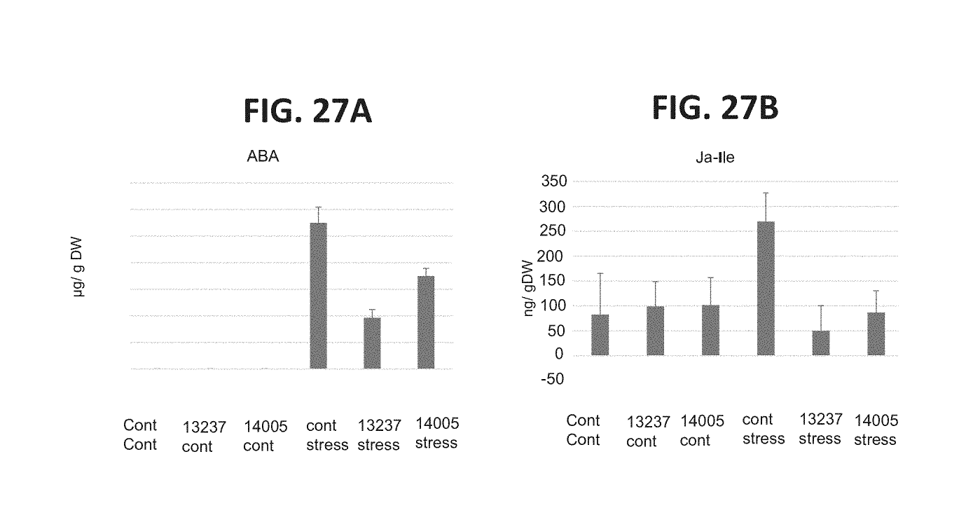

Levels of abscisic acid (ABA) increased dramatically after 10 days of severe drought stress (FIG. 27A). While in well-watered plants ABA remained below detection levels, control stressed plants accumulated 5500 ng of ABA per g of dry leaf. However, levels observed in inoculated plants were significant lower, showing an accumulation of 2000 ng gDW in plants inoculated with Acremonium and 3500 ng gDW in plants inoculated with Sarocladium.

Jasmonic isoleucine levels were significantly induced only in non-inoculated stressed plants whereas no significant differences were observed between well watered controls and inoculated and stressed plants (FIG. 27B).

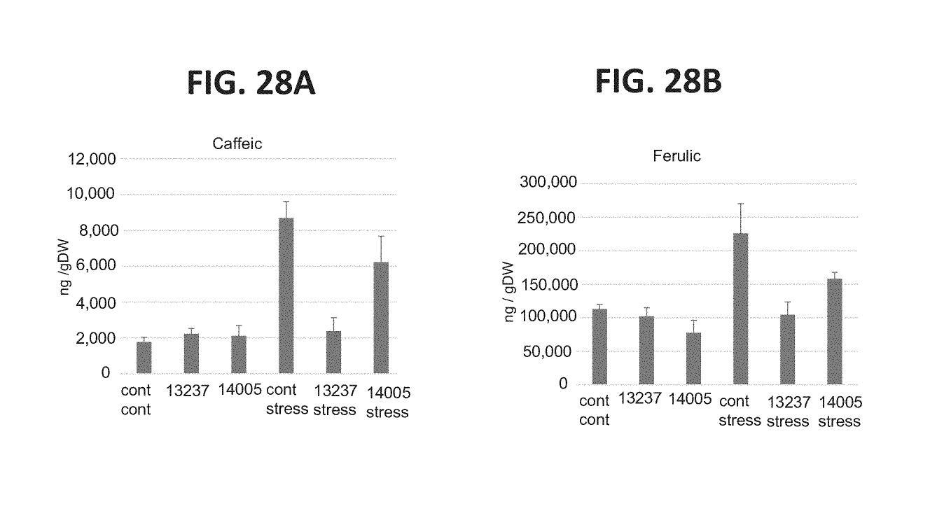

Total phenolic compounds in the samples of leaves subjected to drought stress were higher than in the control samples. Results showed a significant increase of Cafeic and Ferulic acids in wheat leaves after drought stress (FIG. 28). Both phenolic compounds showed a significant increment only in non-inoculated, stressed plants, whereas the stressed plants inoculated with Sarocladium also displayed a moderate increment of Caffeic and Ferulic levels. On the other hand, no significant differences were observed between well-watered controls and stressed plants inoculated with Acremonium.

### Example 5

### Positive Effects on Stress Tolerance in Arabidopsis thaliana

Materials and Methods

Arabidopsis Inoculation and Plant Growth:

Isolates 13237 and 14005 were cultured in Erlenmeyer flasks containing 150 mL of Potato Dextrose Broth medium, which were incubated at 27° C. under agitation at 180 revs min-1 for 7 days. Conidia were collected from 7-day-old PDB cultures by filtration through two layers of Miracloth (Calbiochem) and adjusted to a concentration of 10⁶ conidia/ml with water. Then, A. thaliana seeds cv. Columbia were selected and soaked in conidia suspension (inoculated) or in sterile water (control). After two hours, seed were planted in 100 ml pots containing a peat-based soil. The pots were irrigated with 20 ml of distilled water during the first week and then with 20 ml of nutrient Hoagland solution modified for Arabidopsis every four days during four weeks.

Drought Stress Assay:

Four-week old plants of a similar size were arranged in trays for each treatment. The stress was applied by stopping the irrigation, whereas control plants were maintained with the irrigation regime described above. After 10 days, when controls under drought stress showed wilting symptoms, pictures and samples were taken. In order to determine the dry weight, above ground parts (rosette) were cut, dried in oven for 72 h at 65° C. and weighed.

Chromatographic Analysis:

For hormone and phenolic compound analysis, fresh material was frozen in liquid nitrogen. Dry tissue (0.05 g) was immediately homogenized in 1 ml of ultrapure water and a mixture of internal standards was added prior to extraction (100 ng of [2H6]-ABA, 100 ng of prostaglandin B1, dihydrojasmonic acid, and propylparaben). The extraction and experimental procedures were performed as described by Flors et al. Following extraction, a 20-μl aliquot was directly injected into the UPLC system. Analyses were carried out using a Waters AQUITY UPLC system (Milford, Mass., U.S.A.) with nucleosil ODS reversed-phase column (100 by 2 mm i.d.; 5 μm) (Scharlab, Barcelona, Spain). The chromatographic system was interfaced to a Quatro LC (quadrupole-hexapolequadrupole) mass spectrometer (Micromass, Manchester, U.K.).

Results

Draught Assay:

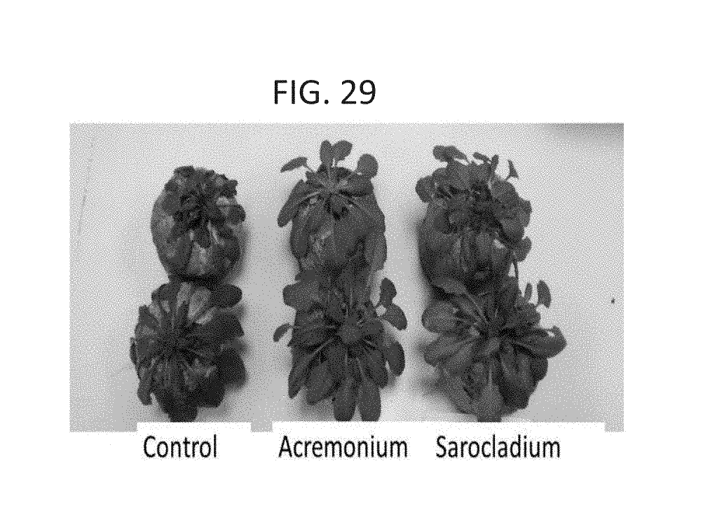

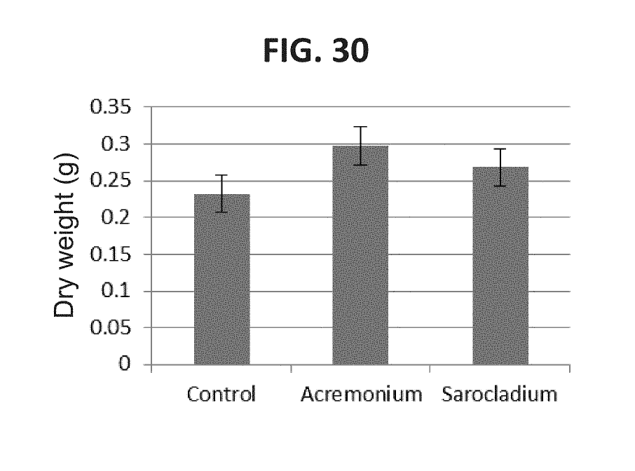

Arabidopsis control plants under drought stress showed wilting symptoms 10 days after stopping the irrigation. Plants inoculated with either Acremonium or Sarocladium endophytes did not show wilting symptoms at this point (FIG. 29). Similar results were obtained in several additional experiments. The dry weight of the plants was 26% higher in Acremonium treated plants and 15% higher in Sarocladium treated plants compared with control plants (FIG. 30).

Measurement of Changes in Stress Metabolites:

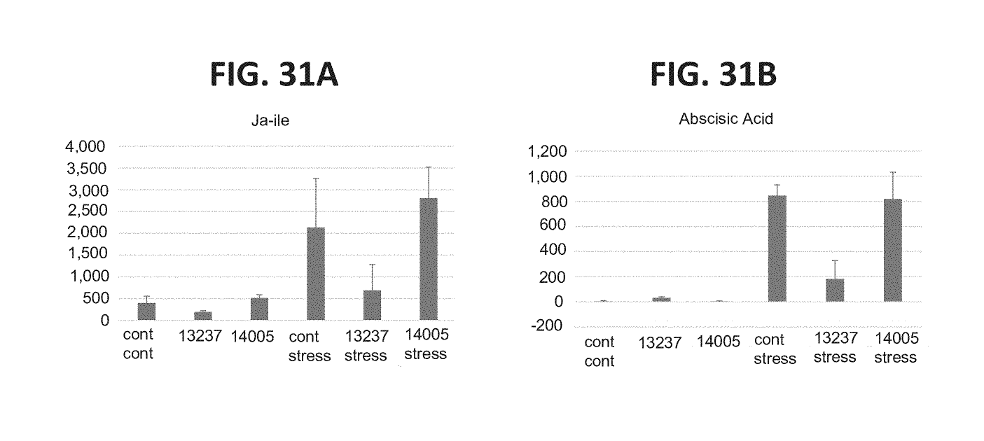

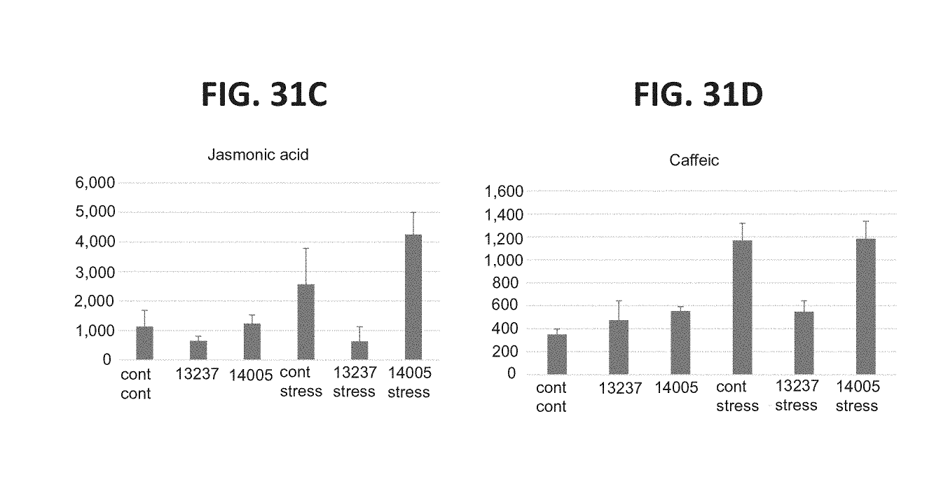

Control plants and Sarocladium inoculated plants under drought regime showed strong accumulation of stress related metabolites such as Abscisic acid, Jasmonic acid, jasmonic-Isoleucine and Caffeic acid. In contrast, plants treated with isolate 13237 (Acremonium) did not accumulate these metabolites and their levels under stress remained similar to levels in well-watered controls FIGS. 31A-D).

### Example 6

### Positive Effects on Additional Plants

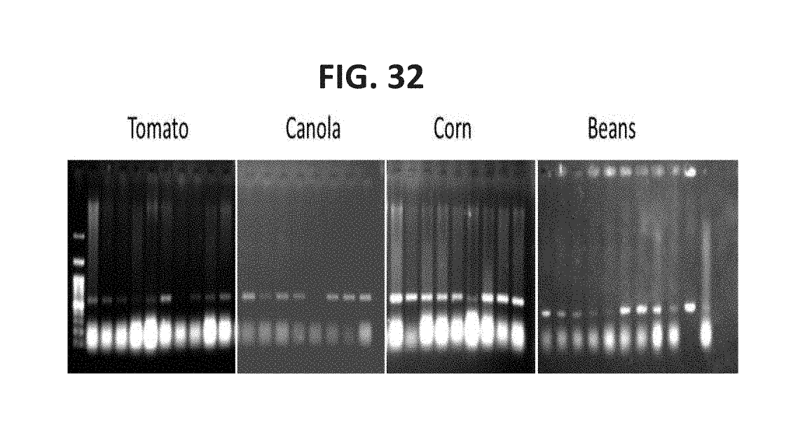

Seeds of corn, canola, tomato, and beans were inoculated with spores of the two endophytes. Plant tissue was taken after 10 days, surface sterilized and presence of the fungus was examined using isolate-specific primers. Presence of the fungus was detected in all cases (FIG. 32).

Induced Draught Tolerance in Maize:

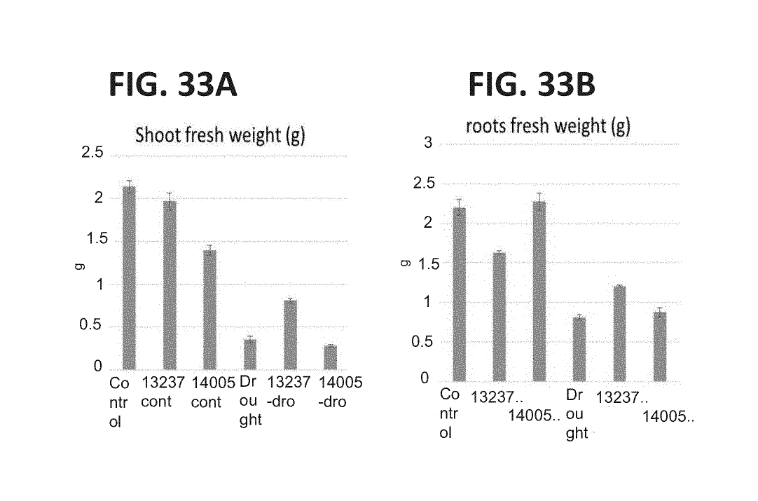

Maize seeds were inoculated with spores of Acremonium or cerocladium. Plants were grown with optimal water until 10 days and then water supply was stopped. Fresh weight of root and shoot were measured (FIG. 33).

### Example 7

### Additional Methods of Inoculation

Infection Through Leaves:

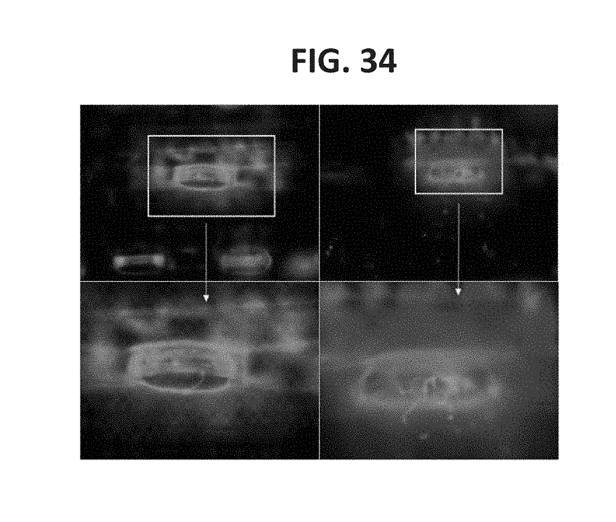

Wheat plants were inoculated by spraying leaves with spores of Acremonium or Serocladium transgenic strains expressing GFP. After 3 days images were taken using a fluorescent microscope. FIG. 34 shows GFP-labelled fungi growing in the leaf tissue and emerging from the stoma. Lower images show enlargement of the parts marked with a rectangle in the top images.

Infection from the Soil:

Killed barley seeds were coated with spores of either Serocladium or Acremonium. The seeds were mixed with soil and added to boxes with sand. Wheat seeds were planted into the soil and grown for 40 days. Then roots and leaves were sampled and presence of the fungus was determined by PCR using species-specific primers. The Acremonium was detected in roots and leaves of the wheat plants.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

Cenis, J. L. (1992). Rapid extraction of fungal DNA for PCR amplification. Nucleic Acids Res, 20(9), 2380.
Crous P W, Petrini O, Marais G F, Pretorius Z A, Rehder F (1995). Occurrence of fungal endophytes in cultivars of Triticum aestivum in South Africa. Mycoscience 36: 105-111.
Edgar, R. C. (2004). MUSCLE: multiple sequence alignment with high accuracy and high throughput. Nucleic Acids Research, 32(5), 1792-1797.
Gardes, M., & Bruns, T. (1993). ITS primers with enhanced specificity for basidiomycetes—application to the identification of mycorrhizae and rusts. Molecular Ecology, 2(2), 113-118.
Ghimire S R and Craven K D (2011). The ectomycorrhizal fungus Sebacina vermifera enhances biomass production of switchgrass (Panicum virgatum L.) under drought conditions. App Environ Microbiol 77: 7063-70767.
Harris, R. S. (2007). Improved pairwise alignment of genomic DNA: ProQuest.
Hubbard M, Germida J J, Vujanovic V (2013). Fungal endophytes enhance wheat heat and drought tolerance in terms of grain yield and second generation seed viability. J Appl Microbiol. In press.
Juneau K. J., Tarasoff C. S. 2012. Leaf area and water content changes after permanent and temporary storage. PLoS ONE 7: e42604.
Kennedy, P. G., Peay, K. G., & Bruns, T. D. (2009). Root tip competition among ectomycorrhizal fungi: Are priority effects a rule or an exception? Ecology, 90(8), 2098-2107. doi: 10.1890/08-1291.1.
Kõljalg, U., Nilsson, R. H., Abarenkov, K., Tedersoo, L., Taylor, A. F. S., Bahram, M., et al (2013). Towards a unified paradigm for sequence-based identification of fungi. Molecular Ecology, 22(21), 5271-5277. doi: 10.1111/mec. 12481.
Larran S, Perelló A, Simón M R, Moreno V (2002). Isolation and analysis of endophytic microorganisms in wheat (Triticum aestivum L.) leaves. World J Microbiol Biotechnol 18: 683-686.
Marshall D, Tunali B (2000). Antagonistic effect of endophytes against several root-rot pathogens of wheat. In: Royo C, Nachit M, Di Fonzo N, Araus J L (eds). Durum wheat Improvement in the Mediterranean Region: New Challenges. Zaragoza: CIHEAM pp 381-386.
Martin, K., & Rygiewicz, P. (2005). Fugal-specific primers developed for analysis of the ITS region of environmental DNA extracts. BMC Microbiology, 5, 28.
Moonsamy, P., Williams, T., Bonella, P., Holcomb, C., Higlund, B., Hillman, G., Daigle, D. (2013). High throughput HLA genotyping using 454 sequencing and the Fluidigm Access Array™ system for simplified amplicon library preparation. Tissue antigens, 81(3), 141-149.
Pa'nka D, West C P, Guerber C A, Richardson M D (2013). Susceptibility of tall fescue to Rhizoctonia zeae infection as affected by endophyte symbiosis. Ann Appl Biol 163: 257-268.
Poling S M, Wicklow D T, Rogers K D, Gloer J B (2008). Acremonium zeae, a protective endophyte of maize, produces dihydroresorcylide and 7-hydroxydihydroresorcylides. J Agric Food Chem 56: 3006-3009.
Porras-Alfaro A, Bayman P (2011). Hidden fungi, emergent properties: endophytes and microbiomes. Annu Rev Phytopathol 49: 291-315.
Redman R S, Sheehan K B, Stout R G, Rodriguez R J, Henson J M (2002). Thermo tolerance generated by plant/fungal symbiosis. Science 298: 1581.
Rodriguez R J White J F, Arnold A E, Redman R S (2008). Fungal endophytes: diversity and functional roles. New Phytol 182: 314-330.
Schloss, P. D., Westcott, S. L., Ryabin, T., Hall, J. R., Hartmann, M., Hollister, E. B., et al (2009). Introducing mothur: open-source, platform-independent, community-supported software for describing and comparing microbial communities. Applied and Environmental Microbiology, 75(23), 7537-7541.
Schulz, B., Wanke, U., Draeger, S., & Aust, H. J. (1993). Endophytes from herbaceous plants and shrubs: effectiveness of surface sterilization methods. Mycological Research, 97(12), 1447-1450. doi: http://dx(dot)doi(dot)org/10.1016/S0953-7562(09)80215-3.
Seifert, K. A. (2009). Progress towards DNA barcoding of fungi. Molecular Ecology Resources, 9(s1), 83-89.
Tanaka A, Takemoto D, Chujo T, Scott B (2012). Fungal endophytes of grasses. Curr Opin Plant Biol 15: 462-468.
Waller F, Achatz B, Baltruschat H, Fodor J, Becker K, Fischer M, Heier T, Hückelhoven R, Neumann C, von Wettstein D, Franken P, Kogel K-H (2005). The endophytic fungus Piriformospora indica reprograms barley to salt-stress tolerance, disease resistance, and higher yield. Proc Natl Acad Sci USA 102: 13386-13391.
White, T., Bruns, T., Lee, S., & Taylor, J. (1990). Amplification and direct sequencing of fungal ribosomal RNA genes for phylogenetics. In M. Innis, D. Gelfand,
J. Shinsky & T. White (Eds.), PCR Protocols: A Guide to Methods and Applications (pp. 315-322): Academic Press.
Zuccaro A, Lahrmann U, Giildener U, Langen G, Pfiffi S, Biedenkopf D, Wong P, Samans B, Grimm C, Basiewicz M, Murat C, Martin F, Kogel K-H (2011). Endophytic life strategies decoded by genome and transcriptome analyses of the mutualistic root symbiont Piriformospora indica. PLoS Pathog 7: e1002290.

1. A composition of matter comprising an agriculturally acceptable carrier and an endophyte which expresses polynucleotides having the sequences as set forth in SEQ ID NOs: 1-5 or an endophyte which expresses polynucleotides having the sequences as set forth in SEQ ID NOs: 6-10.

2. The composition of matter of claim 1, wherein the endophyte which expresses polynucleotides having the sequences as set forth in SEQ ID NOs: 1-5 is deposited under the NRRL deposit No. 67223 or wherein said endophyte which expresses polynucleotides having the sequences as set forth in SEQ ID NOs: 6-10 is deposited under the NRRL deposit No. 67222.

3-4. (canceled)

5. The composition of matter of claim 1, wherein said endophyte is in the form of a spore, a hyphae, or a mycelia.

6. The composition of matter of claim 1, further comprising at least one agent which promotes the growth of a plant.

7-8. (canceled)

9. The composition of matter of claim 1, further comprising a fertilizer.

10. The composition of matter of claim 1, wherein said endophyte is viable.

11-28. (canceled)

29. A method of enhancing the growth of a plant comprising:
(a) inoculating the plant or a part thereof with an endophyte which expresses polynucleotides having the sequences as set forth in SEQ ID NOs: 1-5 or an endophyte which expresses polynucleotides having the sequences as set forth in SEQ ID NOs: 6-10; and
(b) growing the plant, thereby improving the growth of the plant.

30. A method of providing a plant tolerance to a stressful condition comprising:
(a) inoculating the plant or a part thereof with an endophyte which expresses polynucleotides having the sequences as set forth in SEQ ID NOs: 1-5 or an endophyte which expresses polynucleotides having the sequences as set forth in SEQ ID NOs: 6-10; and
(b) growing the plant, thereby providing the plant tolerance to a stressful condition.

31. A method of increasing nutrient uptake in a plant comprising:
(a) inoculating the plant or a part thereof with an endophyte which expresses polynucleotides having the sequences as set forth in SEQ ID NOs: 1-5 or an endophyte which expresses polynucleotides having the sequences as set forth in SEQ ID NOs: 6-10; and
(b) growing the plant, thereby increasing nutrient uptake in the plant.

32. The method of claim 29, wherein said endophyte which expresses polynucleotides having the sequences as set forth in SEQ ID NOs: 1-5 is deposited under the NRRL deposit No. 67223.

33. The method of claim 29, wherein said endophyte which expresses polynucleotides having the sequences as set forth in SEQ ID NOs: 6-10 is deposited under the NRRL deposit No. 67222.

34-39. (canceled)

40. The method of claim 29, wherein said growing is effected under water limiting conditions.

41. The method of claim 29, wherein said growing is effected under a stressful condition.

42. The method of claim 30, wherein said stressful condition is an abiotic stress.

43. (canceled)

44. The method of claim 29, further comprising analyzing the growth of the plant.

45. The method of claim 29, further comprising harvesting the plant.

46. The method of claim 29, further comprising selecting the plant.

47-53. (canceled)


SEQUENCE LISTING (14 sequences)

SEQ ID NO: 1 - DNA, 505 bp, Artificial sequence
  ITS nucleic acid sequence
accttgagaagtggggggtttaacggcgtggttcaaccgctatcctgccgcgagaggtttaattactgcacggaggagttcgcgagggaaccgccactggatttcagggccagccgccgccaggggcaggctgatccccaacgccaggtcccgcgaacgggtcctgagggttgaaatgacgctcagacaggcatgcccgccagaatactggcgggcgcaatgtgcgttcaaagattcgatgattcactgaattctgcaattcacattacttatcgcattttgctgcgttcttcatcgatgccagaaccaagagatccgttgctgaaagttttgatttatttgcttatgccactcagaaatacactaaaagacaagagtttggagcctccggcggacgcctgggtccgggccgcgcgaacgcgcccggggcgaggccgccgaagcaacagtggtaggttcacaatggtttgggagtttttacactcggtaatgatccctccgcagg

SEQ ID NO: 2 - DNA, 573 bp, Artificial sequence
  LSU nucleic acid sequence
  misc_feature (3)..(3): n is a, c, g, or t
ccncagggattgccctagtaacggcgagtgaagcggcaacagctcaaatttgaaatctggcctcacggtccgaattgtaatttgtagaggatgtttctggcgacgtgtcttccgagttccctggaacgggacgccatagagggtgagagccccgtccggtcgtacacctagcctctgtgaaactccttcgacgagtcgagtagtttgggaatgctgctctaaatgggaggtatacgtcttctaaagctaaataccggccagagaccgatagcgcacaagtagagtgatcgaaagatgaaaagcactttgaaaagagggttaaatagtacgtgaaattgctgaaagggaagcgcttatgaccagacttgggctcggtgaatcatccggcgttctcgccggtgcactttgccgtcccaggccagcatcagttcgcgccgggggataaaggtttcgggaatgtagctccctcgggagtgttatagcccgttgcgtaataccctggcgtggactgaggtccgcgctctgcaaggatgctggcgtaatggtcatcagtgacccgtcttgaaacacgga

SEQ ID NO: 3 - DNA, 889 bp, Artificial sequence
  EF-1 nucleic acid sequence
  misc_feature (12)..(12): n is a, c, g, or t
  misc_feature (818)..(818): n is a, c, g, or t
  misc_feature (838)..(839): n is a, c, g, or t
  misc_feature (844)..(845): n is a, c, g, or t
  misc_feature (852)..(852): n is a, c, g, or t
  misc_feature (858)..(858): n is a, c, g, or t
  misc_feature (864)..(864): n is a, c, g, or t
ctggtacttccnaggccgattgtgctattctcatcatcgccgctggtactggtgagttcgaggctggtatctccaaggatggccagacccgtgagcacgctctgctcgcctacaccctgggtgtgcgtcagctcattgttgccatcaacaagatggacaccacccagtggtctgaggcccgttaccaggaaatcatcaaggagacttccaacttcatcaagaaggtcggcttcaaccccaagactgttgccttcgtccccatctctggtttccacggcgacaacatgttggctgcttccaccaactgcccctggtacaagggctgggagaaggagggcaagggtggcgccaaggtcaccggcaagaccctgcttgaggccatcgactccgtcgagccccccaagcgtcccaacgacaagcccctccgtcttcctctccaggatgtctacaagatcggtggtattggcacagtgcccgtcggccgtatcgagaccggtatcctcaagcccggcatggtcgtcaccttcgctcccgccaacgtcaccactgaagtcaagtccgtcgagatgcaccacgagcagctgcccgagggtctccccggtgacaacgttggcttcaacgtgaagaacgtttccgttaaggatatccgccgtggcaacgtcgcctctgactccaagaacgaccccgccatgggcgctgcttccttcaacgcccaggtcattgtcctgaaccaccctggtcaggtcggtgccggttacgctcccgtcctcgactgccacaccgcccacattgcttgcaagttctccgagatcctcganagatcgaccgccgtaccggnngtcgnngagacancccaanttcatnaagtccggcgactctgccatcgtca

SEQ ID NO: 4 - DNA, 882 bp, Artificial sequence
  RBP-2 nucleic acid sequence
  misc_feature (828)..(828): n is a, c, g, or t
  misc_feature (839)..(839): n is a, c, g, or t
  misc_feature (860)..(860): n is a, c, g, or t
  misc_feature (862)..(862): n is a, c, g, or t
  misc_feature (869)..(870): n is a, c, g, or t
gtattacggggcgactagacgcaatattagtgagtaacctcaaggtctggtaacggagatacatacctggttatgatcagggaacgggatgatgctggcgcagataccaagaatcatactgggatgaatctcgcagtgggtgtacatgtgcgtcgttgggttagtctttgtcttgagtcgcttgttgggctcggcttccacgttgtgttcctggggtttctgatccaggtacttctccttgcggtaagactccaaatcttcgggggtcatgcagatcattgccgtctcttcttcttcggcgtccaggtactcgatacacccactcttcacgagatattcccagcccgtggccatctcctccctctcctcgttgaccagatgccagtcatccaacccggcaagatgctccttggtcatgcataacgaccccttttcgggcccgtctagcttgtcttcttgctgcactgtgaatactgggcgcatgacacgaccagcatctgtgacgactctgaactctcgatcccgaatgtcgcggatgacggaagcttcgtggttaaggtcaccatagcgccgcagatgcatgatactatctacgagatctttcgggctcgtatgcacaccgacccaggcaccgttgatgaacaccttggtggcgttggggaaccgctggggttcgtactcttcgagcacttccatgccacggttgatgaagtatccgtagagaggttcgctgggtgtgccaacgggaaacgtgacacatcagtgacaggttcttcaccaaaccacaagcttgaccctcgggggtctcggcaggacacacaagacccnaatgggtgttngtgtagctgccgaggcttcgncngctttcnntcacgggccaat

SEQ ID NO: 5 - DNA, 650 bp, Artificial sequence
  GPD nucleic acid sequence
  misc_feature (5)..(5): n is a, c, g, or t
  misc_feature (13)..(13): n is a, c, g, or t
  misc_feature (645)..(645): n is a, c, g, or t
  misc_feature (647)..(648): n is a, c, g, or t
cgatncgtcttcngcacgccgtcgacaccccgacattgaggtcgtcgctgtcaacgaccccttcattgagacgcactacgctgtgagtctactctgcgcccttccttagttgttgcctccccctcagccggcgccgcaccacccgcgtctgcccctccatgagctttttcaggcgcgcgcagcgtttgcccctccatgatatcatgctcggccctcgccgaaaccggccccaggctgcacctcaatcaggacatggacaaataattgctttgagaccagctgctgactattcgcaaaaggcctacatgctcaagtatgactcctcgcacggtattttcaagggcgacgttgcccaggacggcaacgacctgtccgtcaacggcaagaaggtcaagttctacaccgagcgcgaccccgccgccattccctggaaggacaccggcgccgagtacatcgttgagtccaccggtgtcttcaccaccaccgacaaggctgccgctcacttgaagggcggtgccaagaaggtcatcatctccgccccttcggccgatgcccccatgtacgtgatgggtgtcaacgagaagacctacgacggcaaggccgatgtcatctccaacgcttcttgcacaaccaactgcttggcancnncc

SEQ ID NO: 6 - DNA, 507 bp, Artificial sequence
  ITS nucleic acid sequence
ggggggtttcgaggcgtggccgtgccgctctccggtgcgaggtgtgctactacgcaggggaggctgcggcgcgaccgccactgaatttagggggcgggtgccgccggggcggcctccggtccccaacgccgggcccgttccccgcgaggggtgggcccgagggttgaaatgacgctcggacaggcatgcccgccggagtgccggcgggcgcaatgtgcgttcaaagattcgatgattcactgaattctgcaattcacattacttatcgcatttcgctgcgttcttcatcgatgccagagccaagagatccgttgttgaaagttttgattcatttgttttacgggctttcgcccctcagagatacacgatataaattcgagtttgggtgcccccggcggacgcgaggcggcggaaccgcaacgcgctgagcccgccgagggaaacgtaggtatgttcacagtgggttggagagccaaaaggcactctggtaatgatccctccgcaggt

SEQ ID NO: 7 - DNA, 597 bp, Artificial sequence
  LSU nucleic acid sequence
  misc_feature (1)..(2): n is a, c, g, or t
  misc_feature (5)..(6): n is a, c, g, or t
  misc_feature (15)..(16): n is a, c, g, or t
nntgnnacttacgcnncatccgtgccgagcgcgaacctcagtccaccccagggcattacacgacgggctataacactccccgtagggagccacattcccgccgcctttgtcccccggggcaaactgatgctggcctgagctggcagagtgcaccggcgagaacgccggatgttcaaccaagcccaagtctggtcatgaatgcttccctttcaacaatttcacgtactttttaaccctcttttcaaagtgcttttcatctttcgatcactctacttgtgcgctatcggtctctggccggtatttagctttagaagacgtacacctcccatttagagcagcattcccaaactactcgactcgtcgaaggagctttacagaggctcggtggcacgaccgtacggggctctcaccctctatggcgtcccgttccagggaactcggaagtcacctcgccaaaagcatcctctacaaattacaactcggaccttgcggccagatttcaaatttgagctgttgccgcttcactcgccgttactgaggcaatccctgttggtttcttttcctccgcttattgatatgcttaagttcagccgggta

SEQ ID NO: 8 - DNA, 895 bp, Artificial sequence
  EF-1 nucleic acid sequence
  misc_feature (6)..(6): n is a, c, g, or t
  misc_feature (10)..(10): n is a, c, g, or t
  misc_feature (12)..(13): n is a, c, g, or t
  misc_feature (774)..(775): n is a, c, g, or t
  misc_feature (833)..(833): n is a, c, g, or t
  misc_feature (847)..(847): n is a, c, g, or t
  misc_feature (856)..(856): n is a, c, g, or t
  misc_feature (858)..(858): n is a, c, g, or t
  misc_feature (865)..(865): n is a, c, g, or t
  misc_feature (879)..(879): n is a, c, g, or t
  misc_feature (881)..(881): n is a, c, g, or t
  misc_feature (883)..(885): n is a, c, g, or t
  misc_feature (889)..(890): n is a, c, g, or t
  misc_feature (893)..(893): n is a, c, g, or t
ctggtncctncnngccgactgcgctattctcattatcgctgccggtactggtgagttcgaggctggtatctccaaggatggccagactcgtgagcacgccctgctcgcctacaccctcggtgtgcgtcagctcatcgtcgccatcaacaagatggacaccaccaagtggtctgaggcccgttaccaggagatcatcaaggagacctccaacttcatcaagaaggtcggctacaaccccaagaccgtcgctttcgtccccatctctggtttccacggcgacaacatgcttgctgcctccaccaactgcccctggtacaagggttgggagcgtgaggtcaagggcaacaagcagaccggcaagaccctcctcgaggctattgacggtattgagccccccaagcgtccctccgacaagcccctccgtcttcctctccaggatgtctacaagatcggtggtattggaactgttcccgtcggccgtatcgagactggtgtcctcaagcccggtatggtcgttaccttcgctcccgccaacgtcaccactgaagtcaagtccgttgagatgcaccacgagcagctcactgagggtctccccggtgacaacgtcggcttcaacgtgaagaacgtttccgtcaaggatatccgccgtggtaacgttgcctctgactccaagaacgaccccgccctcggtgccgcttctttcgatgctcaggtcatcgtcctcaaccaccccggtcaggtcggtgctggttacgccccggtccttgattgccnncaccgcccacattgcttgcaagttcgccgagatcaaggagagatcgaccgccgtaccngcaagtctgttgangacgccccnantttcatnaagtctggtgactncncnnncgtnnagnat

SEQ ID NO: 9 - DNA, 850 bp, Artificial sequence
  RBP-2  nucleic acid sequence
  misc_feature (746)..(746): n is a, c, g, or t
  misc_feature (759)..(759): n is a, c, g, or t
  misc_feature (795)..(796): n is a, c, g, or t
  misc_feature (802)..(804): n is a, c, g, or t
  misc_feature (819)..(820): n is a, c, g, or t
  misc_feature (828)..(829): n is a, c, g, or t
  misc_feature (832)..(832): n is a, c, g, or t
  misc_feature (835)..(835): n is a, c, g, or t
  misc_feature (838)..(838): n is a, c, g, or t
  misc_feature (845)..(845): n is a, c, g, or t
  misc_feature (847)..(848): n is a, c, g, or t
  misc_feature (850)..(850): n is a, c, g, or t
gtattacgaggaactgatcgacaagttagctgttgttatcttgatcggtggctcatgagctggcattgcaaaactcacctggttgtgatcagggaaggggatgatgctggcgcaaatacccaagatcatgcttgggtgaatctcgcagtgtgtataggtgtgcgtggtagggttcgtctttgtcttcagtcgcttgttgggatcgtctgccggatcatcttcgggcacttgaactccagacttctgaagtctgtaaatctccaggtcttctggggtcatgcagatcattgccgactcttcttcctcggcatctagatactcgatggcgccactccctgtcaggtcgtcccatgagagaggtgtttggccaagctctgcagcgcgctccttgtctactttgttgaccatgtccttggtgaggatcaaagtgcccttctcgatcccagcgtctggatcatgattttgctcgaccgtgaagaccggtcgcatcacacgacctgcgtccgcgaaaaccttgaactcgcggtctcggatgtcgtgcacaagtgacacctcaggagggatccaacccctccgacgtgtcttgacgagagcgtccaccaacgccttgggctccgaagaaagaccgcaccatgtaccgttgacaaagattttggtatagtctgctatgtgagtgacttcgacatcagcctccctccagcaggtccataccgcggtcgatccaggaacttctcgaatggggttgngcggagatcccanactgacgcaccacatgcaccgaagattcttggaccnnaccgcnnncctgtccttctgganncctccggnnggncanaanaaggccnanntn

SEQ ID NO: 10 - DNA, 744 bp, Artificial sequence
  Beta- tubulin  nucleic acid sequence
  misc_feature (3)..(3): n is a, c, g, or t
  misc_feature (5)..(5): n is a, c, g, or t
  misc_feature (7)..(8): n is a, c, g, or t
  misc_feature (11)..(13): n is a, c, g, or t
tgntnanncannnacgaggtagttcaggtcaccgtaagaggggttggacagcttgagggtgcgcatgcagatgtcgtagagggcctcgttgtcaatgcagaaggtctcgtcggagttctcgacgagctggtggacggagagggtggcgttgtaaggctcaacaacggtgtcggagactttgggggaggggacgacggagaaggtggccatcatgcggtcggggaactcctcacgaatcttggagataaggagggtacccataccggcaccggtaccaccaccgagagagtgggtgatctggaaaccctgaaggcagtcgcagccctcagcctcgcgacggacaacatcgaggacgttgtcgacgagctcggcaccctcagtgtagtggcccttggcccagttgttgccagcaccggactgaccgaagacgaagttgtcggggcggaagagctgaccgaaaggaccagcgcgaacggcatccatggtaccgggctcaagatcgacgaggacggcgcgaggaacgtacttgttaccggaagcctgaagaggcggtcagtgggtccatatcggctagagacgcgttgaagctatcttgaaggacgaaattcggaacacaaacctcgttgaagtagacgctcatgcgctcgagctggagctcagaagagccattgtagacaccgttgctgtcgagaccgtgctcgccagaaaatggtctgccagaaagcagcaccgattggtttagca

SEQ ID NO: 11 - DNA, 20 bp, Artificial sequence
  Single strand DNA oligonucleotide
gtgcgtcgttgggttagtct

SEQ ID NO: 12 - DNA, 20 bp, Artificial sequence
  Single strand DNA oligonucleotide
cacccagcgaacctctctac

SEQ ID NO: 13 - DNA, 20 bp, Artificial sequence
  Single strand DNA oligonucleotide
gttcgaggctggtatctcca

SEQ ID NO: 14 - DNA, 20 bp, Artificial sequence
  Single strand DNA oligonucleotide
gaggacgatgacctgagcat